(19) United States
(12) Patent Application Publication
(10) Pub. No.: US 2023/0032623 A1
(43) Pub. Date: Feb. 2, 2023

(54) Tumor-on-a-chip

(71) Applicant: NEW YORK UNIVERSITY, New York, NY (US)

(72) Inventors: Weiqiang Chen, New York, NY (US); Matija Snuderl, New York, NY (US); Chao Ma, New York, NY (US)

(21) Appl. No.: 17/816,152

(22) Filed: Jul. 29, 2022

Related U.S. Application Data
(60) Provisional application No. 63/227,704, filed on Jul. 30, 2021.

(51) Int. Cl. G01N 33/50; C12M 3/00; C12M 3/06; C12M 1/00

(52) U.S. Cl. CPC G01N 33/5011; C12M 21/08; C12M 23/16; C12M 23/42; C12M 29/10

(57) ABSTRACT

The present invention provides devices that replicate tumor microenvironments in a microfluidic chip. The devices can be used to model certain disease states related to tumor microenvironments. The devices can be adapted to replicate tumor microenvironments from patient-specific cells such that treatment conditions can be modeled and tailored to individual patients. In some embodiments, the devices are suitable for evaluating cancer therapies on a patient-specific basis.

### CROSS REFERENCE TO RELATED APPLICATIONS

This application claims priority to U.S. Provisional Application No. 63/227,704, filed Jul. 30, 2021, which is hereby incorporated by reference herein in its entirety.

### STATEMENT REGARDING FEDERALLY SPONSORED RESEARCH OR DEVELOPMENT

This invention was made with government support under Grant No. R21EB025406 and Grant No. R35GM133646 awarded by the National Institutes of Health. The government has certain rights in the invention.

### REFERENCE TO A “SEQUENCE LISTING,” SUBMITTED AS AN XML FILE

The Sequence Listing, submitted electronically via Patent Center, written as an XML formatted sequence listing with the file name:
“206256-0067-00US Sequence Listing.XML”; created on Jul. 25, 2022, and 13,334 bytes in size, is hereby incorporated by reference.

### BACKGROUND OF THE INVENTION

The lack of robust clinical tools and biomarkers to characterize the heterogeneity and mechanistic details of tumor microenvironments (TME), immune-privileged spaces, which can reliably predict the immunotherapy efficacy, remains a clinical challenge in neuro-oncology. This difficulty stems from not only the limitations of conventional measurement methods, but also the intrinsic heterogeneity and complexity of tumor immunity. Such immune phenotypic “plasticity” and microenvironmental “heterogeneity” are emerging as potential barriers towards effective immunotherapeutic intervention, thus require a patient-specific biomimetic model to test and guide the treatment for individual patients before the start of adjuvant therapy.

Discrepancies between preclinical and clinical results have raised concerns about the predictive value of current animal and patient explant culture models and how the findings from the animal models can be translated to patients. While patient-derived xenografts and explant cultures are considered as the gold standard in preclinical validation, there are significant limitations such as lack of accurate humanized immunity and spatiotemporal evolution of tumor niche interactions. In vitro bioengineering approaches and tumor-on-a-chip strategies can provide additional high-throughput low-cost avenue to test novel therapies and perform patient screening. A few recent 3D tissue engineering approaches with microfluidics and 3D bioprinting have been able to model human tumor stromal microenvironments, or patient-derived tumor organoids with human immune component. While these methods have a clear advantage for high-throughput and clinical relevance, establishing an orthotopic tumor microenvironment for molecularly-distinct tumor subtypes to interrogate the dynamic patient-specific tumor-immune interactions in response to immunotherapy remains a challenge.

There are currently no reliable clinical tools and biomarkers for dissecting the TME-associated mechanisms of therapy resistance and accurately predicting patient response to immunotherapy. Thus, there is a need in immuno-oncology research and clinical management for a truly personalized tissue-engineered biomimetic tumor niche model that allows for an accurate dissection of the microenvironmental heterogeneity and a rapid preclinical testing and screening of personalized immunotherapy. The present invention meets this need.

### SUMMARY OF THE INVENTION

In one aspect, the present invention relates to a tumor-on-a-chip device, comprising: a cartridge housing; a central chamber embedded in the cartridge housing; and a first plurality of evenly spaced micropillars arranged in a substantially circular shape within the central chamber and a second plurality of evenly spaced micropillars arranged in a substantially circular shape within the first plurality of evenly spaced micropillars, such that the central chamber is partitioned into at least an outer region, a middle region, and an inner region; wherein each of the outer region, middle region, and inner region is fluidly connected to at least one aperture; and wherein the outer region comprises endothelial cells configured to mimic a microvasculature, and the middle region comprises tumor cells configured to mimic a tumor. In one embodiment, the inner region is configured for media perfusion and waste removal.

In one embodiment, each of the cells is an autologous cell. In one embodiment, the first plurality of evenly spaced micropillars and the second plurality of evenly spaced micropillars are concentric. In one embodiment, each micropillar of the first and the second plurality of evenly spaced micropillars has a cross-sectional shape selected from the group consisting of: circular, ovoid, square, rectangular, triangular, trapezoidal, and polygonal. In one embodiment, the first and the second plurality of evenly spaced micropillars are evenly spaced by a distance between about 50 μm and 200 μm. In one embodiment, the device further comprises one or more sensors comprising capture molecules or probes positioned within the central chamber. In one embodiment, each of the capture molecules or probes is selected from the group consisting of: antibodies, antibody fragments, antigens, proteins, nucleic acids, oligonucleotides, peptides, lipids, lectins, inhibitors, activators, ligands, hormones, cytokines, sugars, amino acids, fatty acids, phenols, and alkaloids. In one embodiment, each of the one or more sensors is positioned between each of the micropillars.

In one embodiment, the device is configured to replicate or mimic a tumor selected from a cancer consisting of: bladder cancer, bone cancer, brain and spinal cord tumors, brain stem glioma, breast cancer, lung cancer, lymphoma, cervical cancer, colon cancer, colorectal cancer, esophageal cancer, gastrointestinal cancer, hepatocellular (liver) cancer, kidney (renal cell) cancer, melanoma, oral cancer, ovarian cancer, and prostate cancer.

In one embodiment, a device configured to replicate or mimic a brain tumor comprises tumor cells that are glioblastoma cells and further comprises tumor-associated macrophages. In one embodiment, the outer region comprises a population of circulating T-cells.

In one embodiment, a method of determining anti-cancer treatment responsiveness, comprising the steps of: providing the device configured to replicate or mimic a brain tumor; administering an anti-cancer treatment to the device; and determining anti-cancer treatment responsiveness based on a measured change in the device.

In one embodiment, the anti-cancer treatment is a chemotherapeutic selected from the group consisting of: temozolomide, procarbazine, cisplatin, methotrexate, carmustine, lomustine, irinotecan, etoposide, carboplatin, vincristine, and cyclophosphamide. In one embodiment, the anti-cancer treatment is an immunotherapeutic selected from the group consisting of: dinutuximab, pembrolizumab, naxitamab-gqgk, bevacizumab, durvalumab, ramucirumab, cetuximab, nivolumab, and nimotuzumab

In one embodiment, the measured change is a quantity of live and dead tumor cells after 1-3 days treatment or more. In one embodiment, the measured change is high T-cell motility from the outer region into the middle region, indicating more responsiveness to anti-cancer therapy. In one embodiment, the measured change is a polarization of tumor-associated macrophage phenotype towards M2-like phenotype, indicating less responsiveness to anti-cancer therapy. In one embodiment, the measured change is an increase in cytokine levels of TGF-β and/or IL-10, indicating less responsiveness to anti-cancer therapy.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1 depicts a schematic of an exemplary tumor-on-a-chip device.

FIG. 2 depicts a schematic of an exemplary tumor-on-a-chip device seeded with cells to replicate a glioblastoma tumor microenvironment.

FIG. 3A through FIG. 3D depict distinct systemic immunosuppression in clinical GBM patients. (FIG. 3A) A schematic illustrating the stratification of genetic, molecular and cellular characteristics in distinct GBM subtypes. (FIG. 3B) Immunohistochemical analysis of PD-L1 expression on GBM tumors and CD163+ expression on TAM infiltrate. Varied PD-L1 and CD163 expressions with high or no expression in both responding and non-responding GBM patients with administration of a PD-1 inhibitor (nivolumab). Red stars denote brain microvessel. Scale bar is 100 μm. (FIG. 3C) MethylCIBERSORT deconvolution of whole genome DNA methylation data from 435 glioma patients (Capper D et al., Nature. 2018 March; 555(7697):469-74) sorted into six main molecular diffuse glioma subtypes (IDH mutated astrocytoma and oligodendroglioma A_IDH and O_IDH; GBM subtypes Mesenchymal: MES, Proneural: RTK_I, and Classical: RTK_II and RTK_III) shows variability in immune cell subpopulations across GBM subtypes. p-Values for Kruskal-Wallis test are as follows for CD14 (p<2.2⁻¹⁶), CD19 (p<2.2⁻¹⁶), CD4 Eff (p=5.9⁻⁴), CD56 (p=1.2⁻³), CD8 (p<6.9⁻¹⁴), Endothelial (p<2.2⁻¹⁶), Fibroblast (p<2.2⁻¹⁶), Neutrophil (p=2.1⁻¹¹), and Regulatory T-cells (T_{reg}) (p=2⁻¹⁵). CD14 and CD8 were used to identify the monocytic/macrophage and effector T-cell fractions. (FIG. 3D) Clinicopathological information and whole genome DNA methylation showing top 10,000 differentially methylated probes of GBM patients treated with PD-1 inhibitor (nivolumab). Clustering is represented for Responders and non-Responders, irrespective of molecular subtype or other clinicopathological variables (N=9).

FIG. 4 depicts variability in immune cell subpopulations in both responding and non-responding GBM patients with administration of a PD-1 inhibitor (nivolumab). Immune cell subpopulation DNA methylation signatures of patients on PD-1 immunotherapy are deconvoluted using methylCIBERSORT. Noted that Repsonders and who showed no response to nivolumab do not show any significant differences in relative fractions of immune and stromal cells. p-Value of Wilcoxon test for CD14, CD19, CD4 Eff, CD56, CD8, endothelial, fibroblast, neutrophil and regulatory T cells (T_{reg}) is 0.18; 0.39; 0.24; 0.47; 0.9; 0.39; 0.9; 0.12 and 0.27, respectively.

FIG. 5A through FIG. 5J depict modelling the in vivo GBM tumor niche in a ‘GBM-a-on-Chip’ microphysiological system. (FIG. 5A) A schematic diagram illustrating a microfluidics-based GBM-on-a-Chip model to investigate (1) the interactions of immune cell (CD8+ T-cells) with brain microvessels, (2) tumor-associated macrophages (TAMs) and (3) GBM tumor cells in an engineered 3D brain-mimicking ECM. (FIG. 5B) A schematic illustrating the procedures of cell preparation in the microphysiological system. Biomimetic TAMs (CD68+CD163+) were prepared by differentiating monocyte-like U937 cells with 5 nM of PMA for 24 hr, followed by treatments of conditioned-media of GBM cells for 3 days. Simultaneously, fresh allogeneic CD8+ T-cells were isolated from PBMCs and activated and expanded for 3 days with IL-2. (FIG. 5C) Representative confocal immunofluorescence images showing a 3D brain microvessel lumen (yellow) in contact with CD8+ T-cells (green) and GBM (PN, GBML20) tumor cells (red). Scale bar is 50 μm. (FIG. 5D) Representative time-lapsed images showing a single CD8+ T-cell extravasating through brain microvessels (yellow, 0-1 hr), infiltrating through ECM (1-4 hr), and interacting with GBM tumor cells (red, 4-6 hr). Scale bar is 50 μm. (FIG. 5E) Quantified CD8+ T-cell migration speed at different time points of infiltration, indicating the relatively maximum migration speed after extravasation and before contacting with GBM cells. (FIG. 5F) Representative immunofluorescence images showing the distinct counts of allogeneic CD8+ T-cell infiltrate in the PN (GBML20), CL (GBML08) and MES (GBML91) GBM subtypes in GBM-on-a-Chip after 3 days' culture. Note that CD8+ T-cells (green) were in contact with brain microvessels (yellow), TAMs (blue) and GBM tumor cells (red). Scale bar is 50 μm. (FIG. 5G) Quantified results showing more infiltrated allogeneic CD8+ T-cells in the PN GBM as compared to the CL and MES GBMs. (FIG. 5H) Migration trajectories of infiltrated CD8+ T-cell (n>20) for 2 hr in different GBM subtypes. (FIG. 5I) Quantified migration speed of infiltrated CD8+ T-cell, showing faster migration speed in the PN GBM as compared to the CL and MES GBMs at the observation window. Note that the speed range (0-6 μm/min) represents different infiltration stages of different T-cells. (FIG. 5J) Quantified GBM cell apoptosis ratio with the presence or absence of IL activated allogeneic CD8+ T-cell in different GBM niches based on caspase-3/7 activation. Error bars represent ±standard error of the mean (s.e.m.). p-Values were calculated using the Student's paired sample t-test. *, p<0.05.

FIG. 6A through FIG. 6C depicts microfabrication of the microfluidics-based ‘GBM-on-a-Chip’ microphysiological system. (FIG. 6A) A schematic illustrating the layout of ex vivo microphysiological system populated by using patient-resected tumor cells and human primary immune cells. The system consists of peripheral regions (yellow) for brain vascular growth, and immune cell seeding, middle regions (blue) for tumor and stromal TAMs growth, and center region (pink) for cell culture medium infusion. (FIG. 6B) A photo showing the actual microfluidic chip. Scale bar is 5 mm. (FIG. 6C) A schematic demonstrating the synthesis of brain-mimicking HA-rich Matrigel ECM. RGD peptides are conjugated onto Acrylated hyaluronic acid (HA-AC) and crosslinked with MMP-degradable crosslinker (GCRDVPMSMRGGDRCG (SEQ ID NO: 1)). To further mimic the tumor microenvironment growth-factor-reduced Matrigel matrix is interpenetrated with MMP-degradable HA hydrogel for brain tissue-mimicking ECM.

FIG. 7A through FIG. 7D depict sample preparation for TAMs and effector CD8+ T-cells. (FIG. 7A) Representative immunofluorescence images showing CD163 expressions on PMA-treated U937 monocytes with and without treatments of GBML91's conditioned-media. Scale bar is 50 μm. (FIG. 7B) Quantified results showing more CD163+ macrophages in GBML91-educated U937 cells. (FIG. 7C) Quantified flow results showing the purity of sorted CD8+ T-cells from PBMCs. APC represented the fluorescent intensity of CD8+ markers. (FIG. 7D) Quantified results showing the purity of sorted CD8+ T-cells is ˜80%. p-Values were calculated using the unpaired two-tailed Student's t-test. *, p<0.05.

FIG. 8A through FIG. 8C depict CD8+ T-cell extravasation and infiltration behaviors in the engineered GBM microenvironment without the presence of TAM. (FIG. 8A) Quantified results showing similar amounts of extravasated CD8+ T-cells out of vascular in the PN (GBML20), CL (GBML08) and MES (GBML91) GBMs without the presence of TAM. (FIG. 8B) Quantified results showing the migration speeds of infiltrated CD8+ T-cells in all three GBM subtypes without the presence of TAM are comparable. (FIG. 8C) Representative trajectories of infiltrated CD8+ T-cells in the 2 hr observation window in different GBM subtypes without the presence of TAM. Error bars represent ±s.e.m.

FIG. 9A through FIG. 9D depict TAM motility and adherent behaviors in the engineered tumor microenvironments of different GBM subtypes. (FIG. 9A) Representative trajectories of embedded TAM movements in a 2 hr observation window in different GBM subtypes. (FIG. 9B) Quantified results showing faster TAM movement towards the vascular side in the MES (GBML91) GBM as compared to the CL (GBML08) and PN (GBML20) GBMs. Error bars represent ±s.e.m. p-Values were calculated using one-way ANOVA. *, p<0.05. (FIG. 9C) Representative staining images showing different subtypes of GBM-educated TAMs adherent to HBMVECs. TAMs were plated into 24-well plate with HBMVEC monolayer and cultured for 12 hr, followed by 3 times washing with warm media. Scale bar is 200 μm. (FIG. 9D) Number of adherent TAMs to HBMVEC were counted and plotted as cell number per 10⁴ μm² HBMVEC field. p-Values were calculated using unpaired two-tailed Student's t-test, N=30, **p<0.01.

FIG. 10A through FIG. 10G depict distinct systemic immunosuppression in PN, CL and MES GBMs. (FIG. 10A) A schematic highlighting the systemic immunosuppressive signaling among GBM, TAM and CD8+ T-cell via CSF-1/CSF-1R, immunosuppressive cytokines and PD-1/PD-L1. (FIG. 10B) Quantified CD154 and PD-1 expressions (normalized to untreated) on allogeneic CD8+ T-cell, and PD-L1 expression (normalized to untreated) on GBM cells of PN (GBML20), CL (GBML08) and MES (GBML91) subtypes, showing higher expressions of PD-1 and PD-L1 in the MES GBM niche as compared to the PN and CL GBM niches. Surface marker expression was quantified by the mean intensity of each cell. (FIG. 10C) qPCR analysis showing different CD154 and PD-1 expressions in CD8+ T-cell, PD-L1 and CSF-1 expressions in GBM cell. (FIG. 10D) Representative immunofluorescence images showing more immunosuppressive M2-like macrophages in the MES GBM (GBML91) than the PN and CL subtypes. Scale bar is 50 μm. (FIG. 10E) Quantified M2-like marker CD163 expression (normalized to untreated group) on TAM, in different GBM subtypes, showing higher TAM CD163 expression in MES GBM compared to PN (GBML20) and CL (GBML08) GBMs. (FIG. 10F) ELISA results showing high CSF-1 level expressed by MES (GBML91) GBM. (FIG. 10G) Quantified cytokine levels in different GBM derived niches, showing relatively higher expressions of immunosuppressive cytokine (TGF-β1 and IL-10), lower expressions of pro-inflammatory cytokines (IFN-γ and TNF-α) in MES GBM (GBML91). Error bars represent ±s.e.m.,n>80 in B, D, and F. P-values were calculated using one-way ANOVA. *, p<0.05.

FIG. 11A through FIG. 11D depict Analysis of allogeneic CD8+ T-cell activation in various GBM niches. The representative staining images showing (FIG. 11A) CD69, (FIG. 11B) granzyme B (GZMB) and (FIG. 11C) perforin (PFN) expressions on CD8+ T-cells in MES (GBML91), CL (GBML08) and PN (GBML20) GBM niches. Scale bar, 100 μm. The quantified expressions were shown in (FIG. 11D). Noted the relatively lower expressions of T-cell activation markers in the MES GBM as compared to the CL or PN GBM. Surface marker expression was quantified by the mean fluorescent intensity of each cell. Error bars represent ±s.e.m. p-Values were calculated using one-way ANOVA. *, p<0.05, **p<0.01.

FIG. 12A and FIG. 12B depict comparison of cellular and cytokine conditions of IDH-mutant and IDH-wildtype GBM tumor cells. (FIG. 12A) Quantified ratios of CD8+CD154+ cells, CD68+CD163+ cells and caspase-3/7+ apoptosis cells in MGG152 IDH-mutant and proneural GBMs. The PN GBM result represents the mean of three PN GBM cells (GBML20, GBML109, and GS7-11). Note that percentages of CD163+ and apoptosis cells were comparable between the MGG152 and PN GBMs. (FIG. 12B) Comparable concentrations of both pro-inflammatory (TNF-α, IFN-γ) and immunosuppressive (TGF-β and IL-10) cytokines in both cell types. Error bars represent ±s.e.m. p-Values were calculated using the unpaired two-tailed Student's t-test. *, p<0.05.

FIG. 13A through FIG. 13D depict DNA methylation analysis of interactions between patient-derived GBM cell and macrophage in an engineered 3D GBM niche environment. (FIG. 13A) Whole genome DNA methylation analysis showing top 10,000 differentially methylated probes of patient-derived PN (GBML20), CL (GBML08) and MES (GBML91) GBM cells cultured in a 3D brain-mimicking ECM environment with or without macrophages. (FIG. 13B) tSNE analysis of mono-cultured and co-cultured GBM cells showing clear separation of all molecular GBM subtypes, PN (GBML20), CL (GBML08) and MES (GBML91) (each in triplicate) in the same direction when exposed to macrophages. However, the effect appears to be different in the three molecular subtypes with PN GBM mostly affected by presence of macrophage. (FIG. 13C) Whole genome DNA methylation analysis showing top 10,000 differentially methylated probes of mono-cultured and GBM cell-educated macrophages. (FIG. 13D) tSNE analysis of mono-cultured and patient-derived GBM cell-educated macrophages showing distinct shifts in methylation in all molecular GBM subtypes. However, MES GBM cell co-cultured macrophages cluster showed a more distinct separation.

FIG. 14A through FIG. 14E depict top KEGG pathways between mono-cultured and co-cultured GBM cells in a 3D brain-mimicking ECM environment. (FIG. 14A) KEGG pathway analysis from whole genome DNA methylation comparing data from all glioma subtypes co-cultured with macrophages vs all mono-cultured glioma subtypes including PN (GBML20), CL (GBML08), and MES (GBML91) showing activation of pathways involved in axon guidance, Rap1, proteoglycans, and Wnt related signaling. (FIG. 14B) KEGG pathway analysis of macrophages co-cultured with all different types of GBM cells compared with mono-cultured macrophages showed relatively activation of pathways in neuroactive ligand-receptor, Rap1, axon guidance, cAMP related signaling. When DNA methylation data were analyzed for each molecular subtype of GBM, (FIG. 14C) co-cultured PN (GBML20) GBM cells showed relatively higher ratios of genes in proteoglycans, axon guidance, oxytocin, and pluripotency of stem cell related signaling compared to mono-cultured GBM cells, (FIG. 14D) co-cultured CL (GBML08) GBM cells showed relatively higher ratios of genes in PI3K-Akt, axon guidance, focal adhesion, and Rap1-related signaling pathways compared to mono-cultured GBM cells, and (FIG. 14E) co-cultured MES (GBML91) GBM cells showed relatively higher ratios of genes in PI3K-Akt, focal adhesion, chemokine, and actin-related signaling pathways compared to mono-cultured GBM cells.

FIG. 15 depicts PD-L1 promoter methylation in mono-cultured and co-cultured GBM cells in a 3D brain-mimicking ECM environment. The absence or presence of macrophages does not induce changes in methylation of the PD-L1 gene promoter.

FIG. 16A through FIG. 16D depict analysis of extracellular matrix composition in different engineered GBM niches. The quantified results of (FIG. 16A) Collagen IV (Col IV), (FIG. 16B) Fibronectin, (FIG. 16C) Laminin, and (FIG. 16D) hyaluronic acid (HA) depositions in the MES (GBML91), CL (GBML08) and PN (GBML20) GBM niches showed no significant changes in the 3-day culture period. Error bars represent ±s.e.m.

FIG. 17A through FIG. 17H depict targeting TAM with anti-CSF-1R blockade improves anti-PD-1 immunotherapy response in GBM-on-a-Chip. (FIG. 17A) A schematic outlining a dual inhibition therapeutic strategy for targeting both PD-1/PD-L1 and TAM CSF/CSF-1R signaling to inhibit the systemic immunosuppression among GBM, TAM and CD8+ T-cell. (FIG. 17B) Quantified M2-like marker CD163 expression on TAM in response to CSF-1R inhibitor BLZ945 in different GBM subtypes (GBML20, GBML08, and GBML91), showing the limited expression of CD163 in all GBM subtypes. (FIG. 17C) qPCR experiment confirming the inhibition of CD163 expression in TAM with the administration of CSF-1R inhibitor BLZ945. (FIG. 17D) Quantified results showing more infiltrated allogeneic CD8+ T-cells in all GBM subtypes (GBML20, GBML08, and GBML91) with PD-1 and CSF-1R dual inhibition therapy as compared to Nivolumab and BLZ945 monotherapy. (FIG. 17E) Quantified results showing an increased influx of activated CD154+CD8+ T-cells in PD-1 and CSF-1R dual inhibition therapy as compared to Nivolumab monotherapy. (FIG. 17F) Quantified cytokine levels showing significantly increased expression of pro-inflammatory cytokine (TNF-α) and decreased expression of immunosuppressive cytokine (TGF-β1) in most GBM subtypes with dual inhibition therapy. Fold changes were calculated relative to control. Note the patient-specific responses with different pharmacological treatment. (FIG. 17G) Representative apoptosis images showing more apoptotic (green nuclei) GBM cells with co-blockade of PD-1 and CSF-1R relative to control in all GBM subtypes (GBML20, GBML08, and GBML91). Live GBM cells were stained with CellTracker Red (red color). (FIG. 17H) A therapeutic response summary of the heterogeneous and systemic immunosuppression in nine lines of GBM patients' derived cells using GBM-on-a-Chip for relative percentages of GBM cell apoptosis, CD154+CD8+(%) and CD163+CD68+(%) cell populations. 100% stacked bar chart was used to show the relative difference among distinct drug treatments. CSF-1R inhibitor BLZ945 (0.1 μg/ml) and PD-1 blockade nivolumab (1 μg/ml) were used in all the monotherapy or dual inhibition treatments. All control groups were treated with fresh cell culture media supplemented with DMSO (0.01%) and human IgG4 isotype control antibody (1 μg/mL, BioLegend). Error bars represent ±s.e.m. from three independent experiments.n>80 in (FIG. 17B), (FIG. 17D), (FIG. 17E), and (FIG. 17H). P-values were calculated using the Student's paired sample t-test or one-way ANOVA. *, p<0.05.

FIG. 18A and FIG. 18B depict qPCR analysis showing different immunosuppression in TAM and GBM cell. (FIG. 18A) mRNA expression of PD-L1 in GBM-educated TAMs with or without CSF1R inhibition using BLZ945 (0.1 μg/ml). Noted the higher expression in the MES (GBML91) educated TAM relative to the CL (GBML08) and PN (GBML20) educated TAMs. (FIG. 18B) mRNA expression of PD-L1 in GBM cell. Mono-cultured MES (GBML91) GBM expressed more PD-L1 as compared to CL (GBML08) and PN (GBML20) GBMs, which is consistent with the co-cultured GBMs with TAM. Note that CSF-1R inhibition using BLZ945 did not change the PD-L1 expression in GBM cells. Error bars represent ±s.e.m. p-Values were calculated using one-way ANOVA. *, p<0.05.

FIG. 19A and FIG. 19B depict cytokine conditions in different patient-derived GBM cell constructed microenvironments. (FIG. 19A) TNF-α and (FIG. 19B) TGF-β1 concentrations in different GBM microenvironments with single blockade of CSF-1R, PD-1, and co-blockade of PD-1 and CSF-1R. CSF-1R inhibitor BLZ945 (0.1 μg/ml) and PD-1 blockade nivolumab (1 μg/ml) were used in the monotherapy or dual inhibition treatments. Control groups were treated with fresh cell culture media supplemented with DMSO (0.01%) and human IgG4 isotype control antibody (1 μg/mL, BioLegend). Error bars represent ±s.e.m. p-Values were calculated using two-way ANOVA. *, p<0.05.

FIG. 20A through FIG. 20D depict apoptosis ratios of GBM cells under different drug treatments. Quantified apoptosis ratios in (FIG. 20A) control group, (FIG. 20B) BLZ945-treated group, (FIG. 20C) PD-1 blockage group, and (FIG. 20D) dual inhibition therapy of nine patient-derived GBM lines. CSF-1R inhibitor BLZ945 (0.1 μg/ml) and PD-1 blockade nivolumab (1 μg/ml) were used in the monotherapy or dual inhibition treatments. Control groups were treated with fresh cell culture media supplemented with DMSO (0.01%) and human IgG4 isotype control antibody (1 μg/mL, BioLegend). Error bars represent ±s.e.m. p-Values were calculated using one-way ANOVA. *, p<0.05.

FIG. 21A through FIG. 21C depict microglia affect CD8+ T-cell PD-1 expression and GBM cell apoptosis. (FIG. 21A) mRNA expression of PD-1 in CD8+ T-cell in the PN (GBML20), CL (GBML08), and MES (GBML91) GBM niches. Of note, PD-1 expression in CD8+ T-cell co-cultured with GBM, PBMC-derived macrophages and brain-resident microglia was higher than that of those CD8+ T-cells co-cultured with GBM only or GBM and macrophages. (FIG. 21B) Quantified GBM cell apoptosis with the presence or absence of microglia cells in the PN (GBML20), CL (GBML08), and MES (GBML91) GBM microenvironments with CSF-1R inhibitor BLZ945 (0.1 μg/ml), PD-1 blockade nivolumab (1 μg/ml) monotherapy or dual inhibition treatment. Control groups were treated with fresh cell culture media supplemented with DMSO (0.01%) and human IgG4 isotype control antibody (1 μg/mL, BioLegend). These results with the presence of microglia showed no significant difference with the GBM and macrophage only condition. (FIG. 21C) Representative images showing GBM cell apoptosis (green) with or without the presence of microglia cells in the PN (GBML20), CL (GBML08), and MES (GBML91) GBM microenvironments under dual blockages of CSF-1R and PD-1. Scale bar is 100 μm. Error bars represent ±s.e.m. p-Values were calculated using one-way ANOVA. *, p<0.05.

### DETAILED DESCRIPTION

The present invention provides devices that replicate tumor microenvironments in a microfluidic chip. The devices can be used to model certain disease states related to tumor microenvironments. The devices can be adapted to replicate tumor microenvironments from patient-specific cells such that treatment conditions can be modeled and tailored to individual patients. In some embodiments, the devices are suitable for evaluating cancer therapies on a patient-specific basis.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Tumor-On-a-Chip

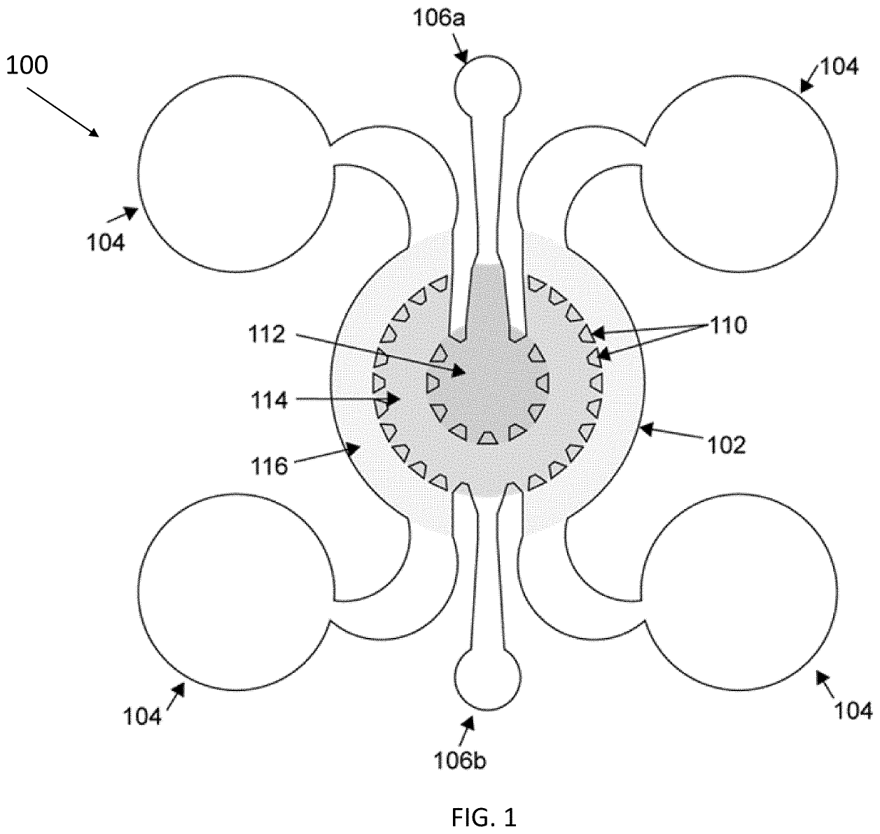

Referring now to FIG. 1, an exemplary layout of a tumor-on-a-chip device 100 is depicted. Device 100 comprises a cartridge housing containing a central chamber 102 fluidly connected to one or more lateral apertures 104 and central apertures 106, wherein each fluid connection comprises one or more microchannels. The housing can be constructed from any desired material and can be at least partially transparent such that central chamber 102 is visible from an exterior of device 100. Central chamber 102 comprises a substantially circular shape and receives one or more cells for co-culture. Central chamber 102 can be subdivided into a plurality of regions, wherein each region receives a population of cells to mimic native tissue architecture. Central chamber 102 can be partitioned into each of the regions by a series of micropillars 110. While micropillars 110 are depicted as having a trapezoidal cross-sectional shape, it should be understood that micropillars 110 can have any desired cross-sectional shape, including but not limited to circular, ovoid, arcuate, square, rectangular, triangular, trapezoidal, polygonal, and the like. In the depicted embodiment, micropillars 110 are regularly spaced by a distance configured to substantially impede flow of viscous materials such as hydrogel solutions, while permitting flow of liquid materials and diffusion of analytes through capillary action. Such a distance can be between, for example, about 50 μm and 200 μm.

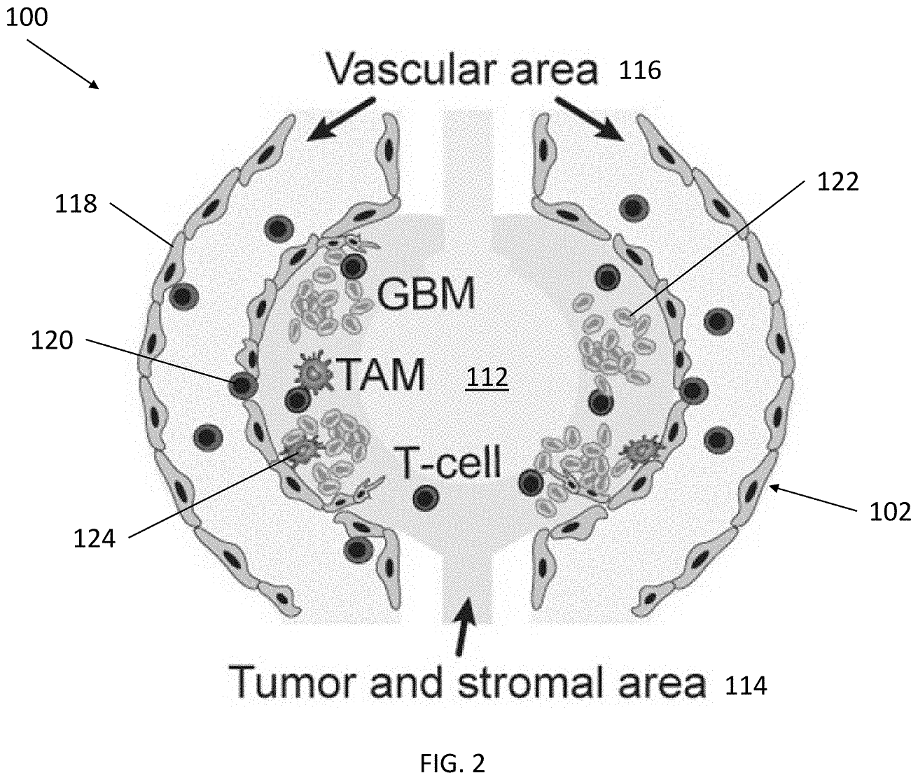

In some embodiments, central chamber 102 can comprise three concentric regions configured to mimic a tumor microenvironment: an outer region 116 representing vasculature, a middle ring region 114 representing a tumor/stromal area, and an inner region 112 for culture medium infusion and waste removal. Each of the regions can be correspondingly seeded with one or more populations of cells. For example, FIG. 2 depicts a glioblastoma model wherein outer ring region 116 is seeded with vascular endothelial cells 118 and through which T-cells 120 are circulated and middle ring region 114 is seeded with glioblastoma cells 122 and tumor-associated macrophages 124. In certain embodiments, apertures of device 100 are preferentially fluidly connected to a partitioned region. For example, FIG. 1 depicts lateral apertures 104 being preferentially fluidly connected to outer ring region 116, central aperture 106a being preferentially fluidly connected to inner region 112, and central aperture 106b being preferentially fluidly connected to middle ring region 114. It should be understood that device 100 can comprise any desired number of apertures in any desired position or arrangement.

In some embodiments, device 100 further comprises one or more sensors for rapid analyte detection. The one or more sensors can comprise any desired sensing mechanism commonly used in art, including but not limited to chemically active regions, electrochemical sensors, immobilized capture molecules, probes, and the like. Contemplated probes or capture agents can be any suitable molecule, including antibodies, antibody fragments, antigens, proteins, nucleic acids, oligonucleotides, peptides, lipids, lectins, inhibitors, activators, ligands, hormones, cytokines, sugars, amino acids, fatty acids, phenols, alkaloids, and the like. The probes or capture agents can be configured to capture any desired molecule, including proteins, amines, peptides, antigens, antibodies, nucleic acids, steroids, eicosanoids, DNA sequences, RNA sequences, bacteria, viruses, and fragments thereof.

The tumor-on-a-chip devices of the present invention can be made using any suitable method known in the art. The method of making may vary depending on the materials used. For example, components substantially comprising a metal may be milled from a larger block of metal or may be cast from molten metal. Likewise, components substantially comprising a plastic or polymer may be milled from a larger block, cast, or injection molded. In some embodiments, the components may be made using 3D printing or other additive manufacturing techniques commonly used in the art. In some embodiments, microstructures and patterns can be achieved through microfabrication techniques including but not limited to: lithography, thin film deposition, electroplating, etching, micromachining, and the like.

### Methods of Modeling Tumors on a Chip

As described elsewhere herein, the tumor-on-a-chip devices of the present invention can be used to model a variety of tumor microenvironments. Accordingly, the present invention further comprises methods of fabricating tumor-on-a-chip devices and methods of characterizing cancer treatment using the tumor-on-a-chip devices.

In some embodiments, tumor microenvironments can be replicated or mimicked by providing tumor cells relevant to a disease or disorder state of interest. Contemplated disease or disorder states include but are not limited to: bladder cancer, bone cancer, brain and spinal cord tumors, brain stem glioma, breast cancer, lung cancer, lymphoma, cervical cancer, colon cancer, colorectal cancer, esophageal cancer, gastrointestinal cancer, hepatocellular (liver) cancer, kidney (renal cell) cancer, melanoma, oral cancer, ovarian cancer, prostate cancer, and the like. In such states, a device 100 can be used to model the progression of a disease or disorder as well as evaluate therapies to treat a disease or disorder.

In some embodiments, a method of the present invention can include a step of providing tumor cells from a source, wherein the source can be a tissue bank, an autologous source, an allogeneic source, or a xenogeneic source. Cells may also be isolated from a number of sources, including, for example, biopsies from living subjects and whole-organs recovered from cadavers. In some embodiments, the isolated cells are autologous cells obtained by biopsy from a subject, such as a cancer patient. Autologous cells can be used in device 100 to model progression and therapy on a patient-specific basis.

Seeding of cells into device 100 may be performed in any desired method. In one embodiment, the cells are embedded in a hydrogel solution and injected into a corresponding region of central chamber 102 by way of the one or more apertures. In certain embodiments, a plurality of cell types are embedded into a hydrogel solution and injected into one or more regions of device 100. In one embodiment, tumor-associated macrophages and tumor cells (such as GBM cells), are both embedded into a hydrogel solution. The plurality of cell types can be embedded into the hydrogel solution at any desired ratio. For example, in one embodiment, the hydrogel is embedded with tumor-associated macrophages and tumor cells (e.g., GBM cells) at an about 1:2 ratio. Injection of hydrogel solution may be accompanied by the application of a gentle vacuum at an oppositely positioned aperture to encourage infiltration of hydrogel solution into a respective region. Contemplated hydrogel solutions include but are not limited to fibrinogen, collagen, hyaluronic acid, alginate, polyacrylamide, polyethylene glycol, and the like. In one embodiment, the hydrogel comprises Matrigel matrix (Corning) and matrix metalloproteinase (MMP)-sensitive hyaluronic acid (HA) hydrogel. In one embodiment, the hydrogel comprises Matrigel matrix (Corning) and matrix metalloproteinase (MMP)-sensitive hyaluronic acid (HA) hydrogels with a volume ratio of about 1:1. The hydrogel solution can be cross-linked within central chamber 102 based the material used, such as by photo-cross-linking, thermal-cross-linking, chemical cross-linking, and the like. In one embodiment, the hydrogel is conjugated with RGD peptides, for examine an RGD peptide comprising the amino acid sequence of SEQ ID NO: 14, or a fragment thereof. In one embodiment, the hydrogel comprises an MMP-degradable crosslinker, such as a crosslinker comprising the amino acid sequence of SEQ ID NO: 1, or a fragment or variant thereof. In various embodiments, the hydrogel solution can be tuned to mimic the extracellular matrix of tissue based on the tumor type being replicated by device 100.

In some embodiments, a method of the present invention can include a step of modifying the provided tumor cells. In some embodiments, a method of the present invention can include a step of providing device 100 seeded with cells as described elsewhere herein to replicate or mimic a tumor microenvironment. The tumor microenvironment can be used to evaluate the effectiveness of anticancer therapies, including but not limited to chemotherapy, radiation therapy, and immunotherapy. Contemplated immunotherapies include but are not limited to: immune checkpoint therapy (such as CDLA4 and PD1 inhibition), adoptive T-cell transfer therapy (such as using autologous or allogenic T-cells in their natural state or in a modified state as in chimeric antigen receptor T-cells), recombinant cancer vaccines, and combinations thereof. Accordingly, in some embodiments a method of the present invention can include a step of applying one or more anti-tumor treatments to a tumor-on-a-chip device and a step of characterizing the effect of the one or more anti-tumor treatments on tumor cells on the tumor-on-a-chip device.

Brain-Tumor-On-a-Chip

As described elsewhere herein, the tumor-on-a-chip devices of the present invention can be used to model a brain tumor microenvironment. In some embodiments, brain tumor microenvironments can be replicated or mimicked by providing brain tumor cells, including but not limited to glioblastomas, meningiomas, astrocytomas, ependymomas, medulloblastomas, oligodendrogliomas, and the like. In some embodiments, a device 100 adapted to replicate or mimic a brain tumor microenvironment can be used to evaluate chemotherapeutic responsiveness, including but not limited to temozolomide, procarbazine, cisplatin, methotrexate, carmustine, lomustine, irinotecan, etoposide, carboplatin, vincristine, cyclophosphamide, and the like. In some embodiments, a device 100 adapted to replicate or mimic a brain tumor microenvironment can be used to evaluate immunotherapeutic responsiveness, including but not limited to dinutuximab, pembrolizumab, naxitamab-gqgk, bevacizumab, durvalumab, ramucirumab, cetuximab, nivolumab, nimotuzumab, and the like. Therapeutic responsiveness can be evaluated over a period of 1-3 days or more. Therapeutic responsiveness can be rated based on the number or percentage of live and dead brain tumor cells, T-cell motility, tumor-associated macrophage phenotype, cytokine levels, and the like.

In some embodiments, device 100 adapted to replicate or mimic a brain tumor microenvironment can be used to identify a patient's glioblastoma subtype (e.g., mesenchymal, proneural, and classical) and responsiveness to certain therapies, such that a method of the present invention includes a step of providing glioblastoma cells from a patient, as well as one or more of human brain microvascular endothelial cells, T-cells, and tumor-associated macrophages from one or more sources (tissue bank, autologous, allogeneic, or xenogeneic as described elsewhere herein), a step of seeding outer ring region 116 with endothelial cells, middle ring region 114 with one or more of glioblastoma cells and tumor-associated macrophages, and circulating T-cells through outer ring region 116. Progression of therapy, such as responsiveness to PD-1 inhibition therapy and/or CSF-1 inhibition therapy, can be assessed by monitoring one or more of T-cell extravasation, migration, activation, expansion, and cytotoxicity.

A method of the present invention can include a step of characterizing anti-cancer therapy in the brain tumor-on-a-chip device. In some embodiments, anti-cancer therapy is evaluated over a period of 1-3 days or more. In some embodiments, anti-cancer therapy responsiveness can be rated based on glioblastoma apoptosis, wherein higher levels of apoptosis indicate trending toward responsiveness to therapy. In some embodiments, anti-cancer therapy responsiveness can be rated based on observed T-cell motility, wherein immobile T-cells indicate trending towards less responsiveness to therapy and highly mobile T-cells indicate trending towards more responsiveness to therapy. In some embodiments, anti-cancer therapy responsiveness can be rated based on tumor-associated macrophage phenotype, wherein tumor-associated macrophages are extracted from the devices and assessed for polarization towards M2-like phenotype, such that a higher percentage of M2-like phenotype indicates trending towards less responsiveness to therapy. In some embodiments, anti-cancer therapy responsiveness can be rated based on cytokine production. For example, increased levels of cytokines including but not limited to TGF-β and IL-10 can indicate trending towards less responsiveness to therapy.

### Tumor-On-a-Chip Kits

The present invention also provides kits for replicating or mimicking tumor microenvironments. The kits include the tumor-on-a-chip devices described elsewhere herein, as well as relevant reagents and instrumentation. For example, in some embodiments, the kit can comprise reagents for loading and culturing cell populations, including but not limited to hydrogels for 3D cell culture, cell culture media, wash media, and the like. In some embodiments, the kit can comprise instrumentation for manipulating contents of the tumor-on-a-chip devices, including but not limited to pipettes, pipette tips, syringes, and the like. In some embodiments, the kit can comprise one or more capture molecules or probes as described elsewhere herein, wherein a user can select the one or more capture molecules or probes for inclusion in the sensors of the bone marrow on a chip devices to detect and/or quantify one or more analytes of interest.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, utilize the present invention and practice the claimed methods. The following working examples therefore, specifically point out exemplary embodiments of the present invention, and are not to be construed as limiting in any way the remainder of the disclosure.

### Example 1: Dissecting the Immunosuppressive Tumor Microenvironments in Glioblastoma-On-a-Chip for Optimized PD-1 Immunotherapy

Glioblastoma (GBM) is the most common and aggressive primary brain tumor among adults, with an average survival of less than 14 months despite aggressive surgery, chemotherapy, and radiotherapy (Stupp R et al., New England Journal of Medicine. 2005 Mar. 10; 352(10):987-96). Programmed cell death protein-1 (PD-1) checkpoint blockade has emerged as a remarkable immunotherapy in pilot GBM clinical trials, yet the durability of patient remission remains largely unpredictable due to heterogeneous tumor immune microenvironments of GBM patients (Cloughesy T F et al., Nature medicine. 2019 March; 25(3):477-86; Di Tomaso T et al., Clinical Cancer Research. 2010 Feb. 1; 16(3):800-13). At most, 8% of GBM patients demonstrate long-term responses in ongoing trials (Reardon D A et al., Neuro-oncology. 2017 Apr; 19 (suppl_3):iii21). However, a lack of clinical biomarkers to predict response represents a critical unmet need to identify potential responders and dissect resistance mechanisms to personalize immunotherapy and combinatorial therapy.

GBM is a genetically heterogeneous disease. Isocitrate Dehydrogenase (IDH)-wildtype GBM tumors can be classified based on genomic, transcriptomic, and DNA methylation data into three main categories, mesenchymal (MES), RTKI/proneural (PN), and RTKII/RTKIII/classical (CL) (Verhaak R G et al., Cancer cell. 2010 Jan. 19; 17(1):98-110). In addition, other molecular subclasses, such as K27M or G34 mutant have recently been recognized (Neumann J E et al., Journal of Neuropathology & Experimental Neurology. 2016 May 1; 75(5):408-14). MES GBM accounts for 30-50% of primary tumors and is associated with particularly poor response to therapy, while PN GBM is associated with a somewhat better prognosis. While some reports have shown an enrichment of PD-L1ww specimens in PN GBM and PD-L1^{HIGH} specimens in MES GBM (Berghoff A S et al., Neuro-oncology. 2015 Aug. 1; 17(8):1064-75), PD-L1 tumor expression has not been shown to directly predict clinical outcomes (Taube J M et al., Clinical cancer research. 2014 Oct. 1; 20(19):5064-74). Molecular GBM subgroups are associated with distinct histological patterns, suggesting that tumor microenvironmental features reflect the specific underlying molecular genetic abnormalities. In addition, GBM contain a highly immunosuppressive tumor microenvironment with abundant tumor-associated macrophages (TAMs), low number of cytotoxic T lymphocytes (CTLs) (Razavi S M et al., Frontiers in surgery. 2016 Mar. 2; 3:11; Nduom E K et al., Neuro-oncology. 2015 Nov. 1; 17 (suppl_7):vii9-14). The role of GBM molecular subtype and impact on tumor immune microenvironment and anti-PD-1 immunotherapy remain poorly understood.

Improving the clinical use of anti-PD-1 immunotherapy in GBM patients requires a comprehensive understanding of tumor genetics and microenvironment as well as the ability to dissect the dynamic interactions among GBM and immune suppressor cells, particularly TAM (Hambardzumyan D et al., Nature Neuroscience. 2016 January; 19(1):20). TAM represents the majority of immune population in GBM (30%-50%), and high TAM density correlates with poor prognosis, and resistance to the therapy (Hambardzumyan D et al., Nature neuroscience. 2016 January; 19(1):20). GBM has been demonstrated to secrete immunosuppressive factors including transforming growth factor-β1 (TGF-β1), and colony-stimulating factor-1 (CSF-1) polarizing monocytes toward an immunosuppressive ‘M2-like’ phenotype (Lu-Emerson C et al., Neuro-oncology. 2013 Aug. 1; 15(8):1079-87; Cui X et al., Biomaterials. 2018 Apr. 1; 161:164-78; Thomas A A et al., Cancer journal (Sudbury, Mass.). 2012 January; 18(1):59). An in silico analysis of immune cell types in patient GBM biopsies found that the M2-TAM gene signature indicated a greater association with the MES subtype (13%) compared to the non MES subtypes: CL (6%) and PN (5%) (Wang Q et al., Cancer cell. 2017 Jul. 10; 32(1):42-56). TAM-targeting agents like CSF-1 receptor (CSF-1R) inhibitor have shown promise by reprogramming M2-TAMs toward an anti-tumorigenic ‘M1’ phenotype in murine glioma models, yet clinical trials on GBM patients showed poor response and patients acquired resistance by the tumor microenvironment (Pyonteck S M et al., Nature medicine. 2013 October; 19(10):1264). While numerous clinical trials are under way to explore combining anti-CSF1R and immunotherapy (Cannarile M A et al., Journal for immunotherapy of cancer. 2017 December; 5(1):1-3), there are no biomarkers that could identify patients who could benefit from such combination.

The inability to predict immunotherapy efficacy and identify therapy resistance mechanisms is a major challenge in immuno-oncology including neuro-oncology (Agrawal N S et al., Journal of neurology and neurosurgery. 2014 Apr. 1; 1 (1)). Discrepancies between preclinical and clinical results have raised concerns about the predictive value of current animal and patient explant culture models and how the findings from the animal models can be translated to patients. While patient-derived xenografts (Xu Z et al., InGlioblastoma 2018 (pp. 183-190). Humana Press, New York, N.Y.; Huszthy P C et al., Neuro-oncology. 2012 Aug. 1; 14(8):979-93) and explant cultures (Shimizu F et al., Current protocols in stem cell biology. 2011 December; 19(1):3-5) are considered as the gold standard in preclinical validation, there are significant limitations such as lack of accurate humanized immunity and spatiotemporal evolution of GBM tumor niche interactions (Binnewies M et al., Nature medicine. 2018 May; 24(5):541-50). In vitro bioengineering approaches and tumor-on-a-chip strategies can provide additional high-throughput low-cost avenue to test novel therapies and perform patient screening. A few recent three-dimensional (3D) tissue engineering approaches with microfluidics and 3D bioprinting have been able to model human GBM tumor stromal microenvironments (Xiao Y et al., Advanced Science. 2019 April; 6(8):1801531; Yi H G, Jeong Y H, Kim Y, Choi Y J, Moon H E, Park S H, Kang K S, Bae M, Jang J, Youn H, Paek S H. A bioprinted human-glioblastoma-on-a-chip for the identification of patient-specific responses to chemoradiotherapy. Nature biomedical engineering. 2019 July; 3(7):509-19; Linkous A et al., Cell Rep 26: 3203-3211. e5), or patient-derived tumor organoids included human immune component (Moore N et al., Lab on a Chip. 2018; 18(13):1844-58). While these methods have a clear advantage for high-throughput and clinical relevant analysis, establishing an orthotopic tumor microenvironment for molecularly distinct GBM subtypes to interrogate the dynamic patient-specific tumor-immune interactions in response to immunotherapy remains a challenge.

The present study integrates critical hallmarks of the immunosuppressive GBM microenvironments in a microfluidics-based ex vivo microphysiological system termed ‘GBM-on-a-Chip’. The system is utilized to identify potential therapy responses in a cohort of molecularly distinct GBM patients. At a single-cell resolution, longitudinal analysis could be performed on allogeneic CD8+ T-cells trafficking through 3D brain microvessels, infiltrating brain-mimicking tissue, and interact with TAMs and GBM cells. By employing cellular (immune cell infiltrate composition, phenotypes, and dynamics), genomic and epigenetic (DNA), transcriptomic (RNA), and proteomic (cytokines) microenvironmental signatures, the immune-regulatory mechanisms of the GBM microenvironment that evoke resistance to PD-1 inhibition were dissected, and co-targeting of PD-1 immune checkpoint and TAM-associated CSF-1R signaling was demonstrated to improve therapeutic efficacy in GBM. Hence, the ex vivo patient-specific ‘GBM-on-a-Chip’ may significantly lead to personalized immunotherapy screening, improving therapeutic outcomes in GBM patients.

The materials and methods are now described.

Patients

All tumor biopsies were molecular profiled using clinically validated next-generation sequencing, MGMT promoter methylation analysis by pyrosequencing and molecularly classified by clinically validated whole genome DNA methylation as described previously (Capper D et al., Nature. 2018 March; 555(7697):469-74). Patients received nivolumab ‘off-label’ for newly diagnosed or recurrent glioblastoma. Nivolumab was administered at 3 mg/kg intravenous injection every 14 days. Median duration of nivolumab therapy was 3.5 months (range 0.5 to 15 months). Patients were assessed clinically once per months and had follow-up MRI assessments every 2 months. Patients were classified as ‘responders’ if they appeared clinically (improving or stable neurological deficits without need for steroids) and radiographically (MM demonstrating <25% increase in abnormal enhancement compared to pre-nivolumab baseline MRI brain) for at least 3 months after beginning immunotherapy. Patients were classified as ‘non-responders’ if they were clinically deteriorating (worsening symptoms, increasing steroid requirements) or if MRI demonstrates >25% increase in contrast-enhancing within 3 months from start of nivolumab therapy.

Patient-Derived Tumor Cells and Culture

Fresh tumor tissues were harvested from GBM patients undergoing resection surgery of GBM after informed consent (IRB no. 12-01130) (Bayin N S et al., Oncogenesis. 2016 October; 5(10):e263), and characterized for different GBM subtypes. Patient-derived cells of GBML08, GBML20, GBML83, GBML91, GBML107, GBML109 and MGG152 were cultured in GBM basal medium supplemented with every 2-3 days with 20 ng/ml Epidermal growth factor (EGF, Sigma-Aldrich) and 20 ng/ml basic fibroblast growth factor (bFGF, Sigma-Aldrich). GBM basal medium was prepared with Neurobasal media (21103049, Gibco), 1× Non Essential Amino Acids (11140-050, Gibco), 1× B27 (without Vitamin A) (12587-010, Gibco), and 1× N2 (17502-048, Gibco). Patient-derived cells of GS7-11, GSC20, and GSC289 were cultured in Dulbecco's modified Eagle's medium (DMEM, Sigma-Aldrich) supplemented with 1× B27 Supplement (17504-044, Gibco), 20 ng/mL EGF (E9644, Sigma-Aldrich), 20 ng/mL bFGF (F0291, Sigma-Aldrich) and 1% penicillin/streptomycin (Gibco). Parental tumors and cultures derived from them were always profiled with DNA methylation arrays and with RNA-sequencing, to ensure maintenance of the molecular subtype.

Cell Culture and Reagents

HBMVECs (10HU-051, iXCells Biotechnologies) were cultured in recommended Endothelial Cell Growth Medium (MD-0010, iXCells Biotechnologies). Cells are collected with 0.05% trypsin-EDTA and subcultured with a plating density of 5×10³ cells/cm². Only early passages of HBMVECs (passage 1-6) are used in the assays. U937 monocytes (ATCC) were maintained in RPMI-1640 medium (Gibco) supplemented with 10% fetal bovine serum (FBS, Gibco) and 1% penicillin/streptomycin (Gibco). Human microglia cell line HMC3 (CRL-3304, ATCC) was cultured in Eagle's Minimum Essential Medium (EMEM, 30-2003, ATCC), supplemented with 10% FBS (Gibco) and 1% penicillin/streptomycin (Gibco). All the cells were cultured in a 37° C. incubator with 5% CO₂. These cell lines have been authenticated with the short tandem repeats (STR) profiling and mycoplasma testing.

TAM and CD8+ T-Cell Preparation

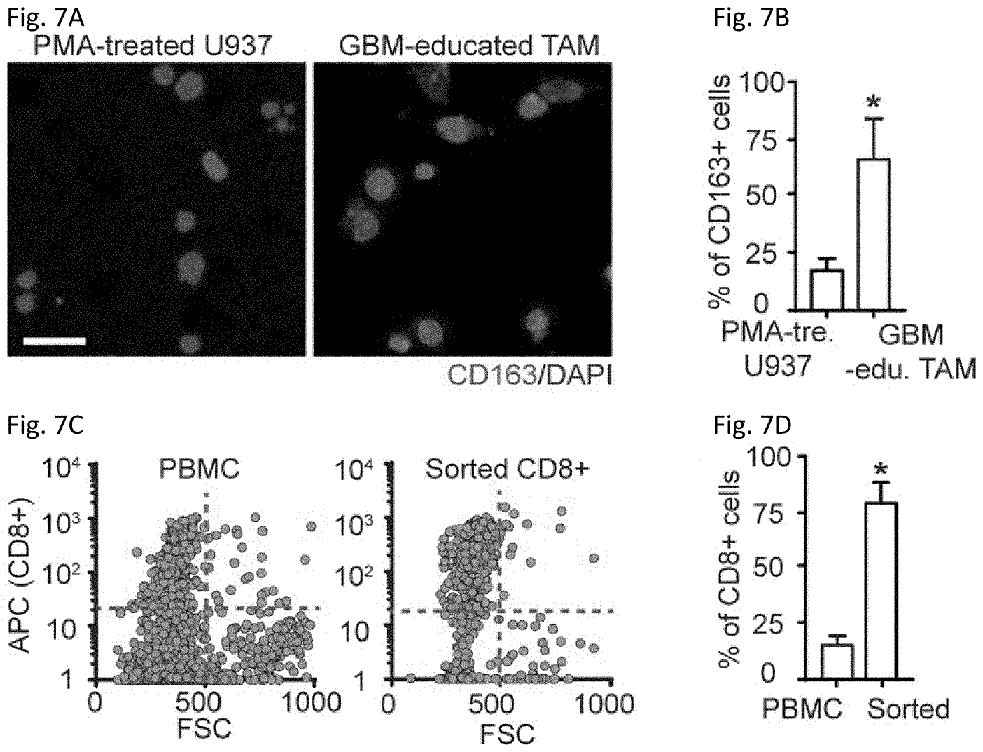

U937 monocytes were polarized into macrophages with treatments of 5 nM PMA (Sigma-Aldrich) for 24 hr (Shi Y et al., Nature communications. 2017 Jun. 1; 8(1):1-7). Biomimetic TAMs (CD68+CD163+) were obtained by culturing U937-derived macrophages in complete culture media supplemented with supernatants of patient-derived GBM cells (5×10⁵ cells/mL) which were collected after 3 days' culture and centrifuged at 2000×g for 10 min at 4° C. to remove cell debris. Cryopreserved human PBMCs (10HU-003, iXCells Biotechnologies) were thawed and cultured in RPMI-1640 medium (Gibco) supplemented with 10% FBS (Gibco) and 1% penicillin/streptomycin (Gibco) overnight before sorting for CD8+ T-cells. Allogeneic CD8+ T-cells were isolated from PBMCs via negative selection using MojoSort Human CD8 T-Cell Isolation Kit (MojoSort, 480011, Biolegend) as per the manufacturer's protocol (FIG. 7A through FIG. 7D). Isolated CD8+ T-cells were activated and expanded for 2-3 days in PBMC culture medium supplemented with 10 ng/mL recombinant IL-2 (589104, Biolegend).

Microfluidic Chip Fabrication

A microfluidic chip containing a set of vascular-seeding channel, hydrogel loading channel, and media infusion channel was used to build the GBM microenvironment. The microfluidic chips were fabricated using the standard soft lithographic method. Briefly, silicon master molds were first fabricated by standard photolithography using SU-8 photoresist (SU8-2075, Microchem) with a thickness of 100 μm. After coating trichloro (1H, 1H, 2H, 2H-perfluorooctyl) silane (448931, Sigma-Aldrich) vapor overnight in vacuum desiccation to facilitate the Polydimethyl siloxane (PDMS, Sylgard 184, Dow Corning) release, PDMS prepolymer was mixed with a curing agent at a weight ratio of 10:1, poured onto the master molds, degassed in a vacuum desiccation for 2 hr to remove air bubbles, and cured at 80° C. overnight. Silicone slabs were then cut out from the master molds and punched to make inlets and outlets for vascular-seeding channels, hydrogel loading channels and media infusion channels. Finally, oxygen plasma (350W, PlasmaEtch) was applied to irreversibly bond PDMS slabs and glass coverslips, and then baked overnight at 80° C.

Synthesis and Preparation of Brain Tissue-Mimicking Hydrogel

Brain tissue-mimicking hydrogel was prepared by interpenetrating growth-factor-reduced Matrigel matrix (Corning) and matrix metalloproteinase (MMP)-sensitive hyaluronic acid (HA) hydrogels with a volume ratio of 1:1. MMP-sensitive HA hydrogel was synthesized as described previously (Wang H et al., Advanced healthcare materials. 2019 February; 8(4):1801234). Briefly, HA-ADH was firstly obtained using hyaluronic acid (100 mg, 0.0015 mmole, 50 kDa), ADH (2.6 g, 8.4 mmole) and 1-ethyl-3-[3-dimethylaminopropyl] carbodiimide hydrochloride (EDC) (0.3 g, 0.92 mmole) at pH 4.75, followed by dialysis (MWCO 6-8 kDa) in deionized water for 2-3 days and lyophilizing. Acrylated hyaluronic acid (HA-AC) was prepared by reacting the synthesized HA-ADH (100 mg, 0.0014 mmole) with N-Acryloxysuccinimide (NETS-AC) (108 mg, 0.47 mmole) in HEPES buffer overnight and dialysis in DI water for 2-3 days before lyophilizing. HA-AC was further conjugated with RGD peptides (Ac-GRGDSPCG-NH2 (SEQ ID NO. 14), Genscript) overnight, dialysis in DI water for 2 days and lyophilizing. Finally, MMP-sensitive HA hydrogel (10 mg/mL) was obtained by crosslinking with MMP-degradable crosslinker (GCRDVPMSMRGGDRCG (SEQ ID NO. 1), Genscript).

Generation of Ex Vivo Tumor Microenvironment

To firmly bond the brain tissue-mimicking hydrogels in the microfluidic chip, fabricated chip was firstly treated with oxygen plasma (350W, 2 min), then coated with 1 mg/mL Poly-D-Lysine (A-003-E, Millipore Sigma) for 2 hr at room temperature. After washing completely with distilled water at least twice, the microfluidic chip was further baked at 80° C. overnight to recover the hydrophobic property of the PDMS channels. The microfluidic chip was then transferred into a cell culture biosafety hood, and subsequently sterilized with UV for 30 min. To avoid contamination, all the following procedures were conducted in the sterile microenvironment.

Patient-derived GBM cells were firstly dissociated with Accutase (Innovative Cell Technologies), and then labeled with CellTracker Red (5 μM, C34552, Thermo Fisher-Scientific) as per the manufacturer's instructions. TAMs and GBM cells were then mixed with a number ratio of 1:2 at a cell density of 1×10⁸ cells/mL in the brain tissue-mimicking hydrogel. The final cell numbers of GBM and TAMs were about 1×10⁵ cells per chip. Notably, cells were first suspended in a HA hydrogel, then loaded into Matrigel on ice to avoid the gelation of Matrigel. Hydrogel solution was then quickly flushed into the hydrogel loading channel, and then incubated in an incubator at 37° C. and 5% CO₂ for 30 min for complete gelation. After incubation, the media infusion channel and the vascular-seeding channel was flushed with fresh cell culture media for 2 hr. HBMVECs labeled with DiD (5 μM, V22887, Thermo Fisher-Scientific) were seeded into the vascular-seeding channel at a density of 5×10⁶ cells/mL, and incubated at 37° C. and 5% CO₂ overnight with fresh cell culture media. To form the vascular lumens, the microfluidic chip was flipped every 15 mins for 2 hr to let HBMVECs to uniformly attach to the vascular-seeding channel. After 24 hr′ incubation, IL-2 activated allogeneic CD8+ T-cells were firstly labeled with CellTracker Green (5 μM, C2925, Thermo Fisher-Scientific) and loaded into the vascular channel at a total cell number of 1×10⁶ cells per chip. After culture in the incubator for 1-3 days, live cell images of different cell compartments were captured by an inverted fluorescent microscope (Zeiss Axio Observer.Z1).

Cell Fixation and Immunostaining

To characterize the expression of surface marks on GBM cell, CD8+ T-cell and TAM, cells were firstly recovered with Corning Cell Recovery Solution (Corning, N.Y., USA) from the brain tissue-mimicking hydrogel. Briefly, cell culture medium was aspirated, and the microfluidics chip was rinsed with cold 1× PBS twice, 10 min per round. PDMS slabs were detached from the cover glass in the microfluidics chip, followed by incubation with Corning Cell Recovery Solution on ice for 30 min. With gently pipetting the channel containing cells, the cell suspension was centrifuged, washed with cold 1 x PBS twice and then resuspended in 5% BSA blocking buffer. All cell samples were fixed in 4% paraformaldehyde (PFA) (Sigma-Aldrich) for 10 min at room temperature. To identify different surface makers on T-cell, TAM, and GBM cell, cells were first stained with specific primary antibodies, and then visualized with secondary antibodies. Specifically, CD8 and CD154 expressions on T-cell were stained with Alexa Fluor 647 conjugated anti-human CD8 (344726, Biolegend) and PE conjugated anti-human CD154 (310805, Biolegend) at 4° C. for 30 min. To quantify PD-1 expression on CD8+ T-cell, cells were first incubated with PD-1 primary antibody (367402, Biolegend) for 1 hr, and then visualized with Alexa Fluor 488 conjugated goat anti-mouse IgG secondary antibodies (Invitrogen, 5 μg/mL). TAMs were identified by staining with Alexa Fluor 647 conjugated anti-human CD68 antibody (333819, Biolegend), followed by staining with anti-human CD163 primary (333602, Biolegend) and Alexa Fluor 488 conjugated goat anti-mouse IgG secondary antibodies (Invitrogen, 5 μg/mL), or PD-L1 primary antibody (MAB1561, R and D Systems) and Alexa Fluor 555 conjugated goat anti-mouse IgG secondary antibodies (Invitrogen, 5 μg/mL). To identify PD-L1 expression on GBM cell, GBM cells were labeled with CellTracker Red (5 μM, C34552, Thermo Fisher-Scientific) before loading into the chip. After recovering cells from the chip, PD-L1 primary antibody and Alexa Fluor 488 conjugated goat anti-mouse IgG secondary antibodies (Invitrogen, 5 μg/mL) were used to stain PD-L1 expression. Fluorescent images of stained cell samples were obtained by an inverted fluorescent microscope (Zeiss Axio Observer.Z1). PD-1 and CD154 expressions on CD8+ T-cell, PD-L1 and CD163 expressions on macrophage were quantified based on the mean florescent intensity of cell staining using ImageJ (NIH). Alternatively, ratios of CD154+ or CD163+-cells were calculated relative to CD8+ cells or CD68+ cells, respectively.

Quantification of Cell Migration

Infiltrated CD8+ T-cells were defined as the CD8+ T-cells migrating out of vascular lumens. To quantify the migration behaviors of those CellTracker Green-labeled allogeneic CD8+ T-cells, time-lapsed image stacks were acquired every minute for 2 hr and at least three positions in each microfluidic chip. The cell centroids at different time points of the same cell were then used to calculate the cell migration speed and linked up to represent the migration trajectories by using ImageJ (NIH). The mean migration speeds were then averaged for all infiltrated cells.

CD8+ T-Cell Activation Analysis

To characterize the effector function of allogeneic CD8+ T-cell, cells in the devices were fixed and permeabilized, followed by stained with PE conjugated anti-human CD154 (310805, Biolegend), FITC conjugated anti-human CD69 (310904, Biolegend), PE conjugated anti-human Perforin Antibody (308106, Biolegend), or PE conjugated anti-human/mouse Granzyme B Recombinant Antibody (372208, Biolegend). Fluorescent images were obtained by a fluorescent microscope (Zeiss Axio Observer.Z1) with a 40× objective, then analyzed using ImageJ (NIH).

Quantification of GBM Cell Apoptosis

To examine the apoptosis of GBM cells in the microfluidic model, CellEvent Caspase-3/7 Green Detection Reagent (R37111, Thermo Fisher-Scientific) and Hoechst 33342 (5 μg/mL; H3570, Thermo Fisher-Scientific) were used to distinguish the apoptotic cells that with activated caspase-3/7 with bright green nuclei. Briefly, the Caspase-3/7 Detection Reagent was diluted in fresh cell culture media as per the manufacturer's instructions and replenished into the microfluidic chip 3 days after forming the GBM niche. After incubation at 37° C. for 1 hr, imaging was conducted immediately by an inverted fluorescent microscope (Zeiss Axio Observer.Z1) with a 10× objective. The ratio of apoptotic GBM cells were calculated as the number of cells with green nuclei to the total number of GBM cells that were stained with CellTracker Red (5 μM, C34552, Thermo Fisher-Scientific).

Cytokine Quantification

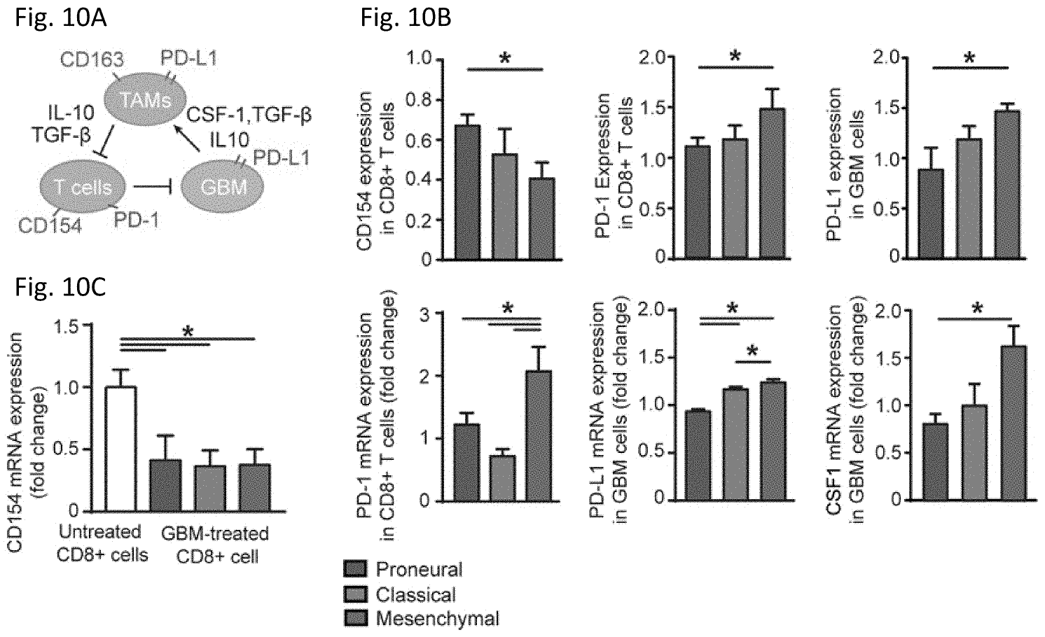

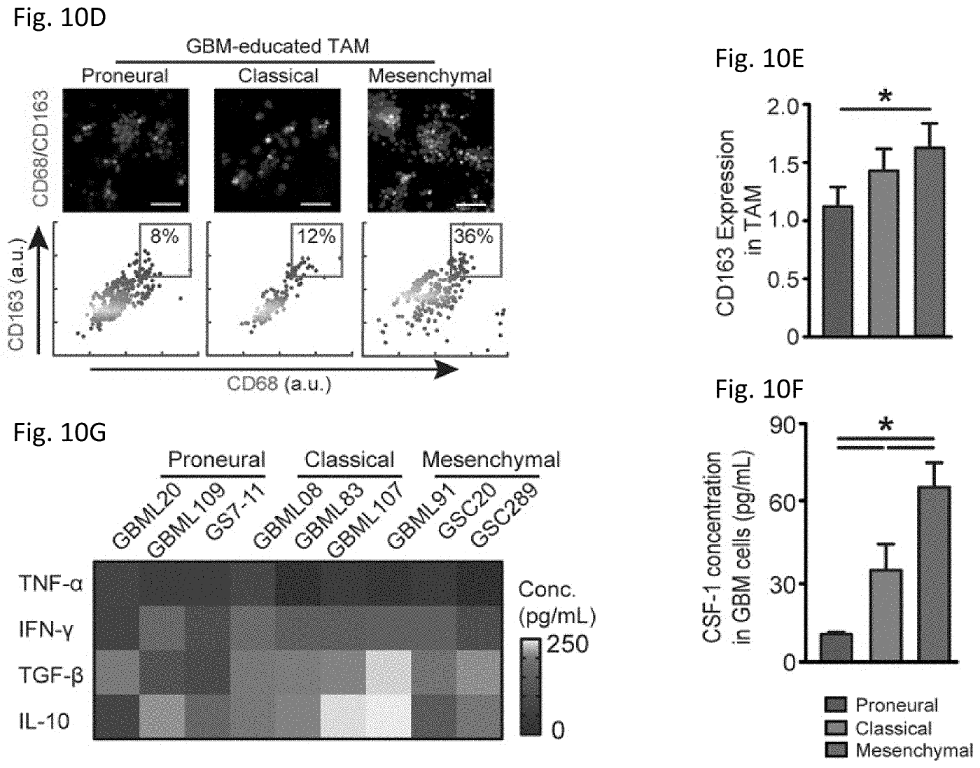

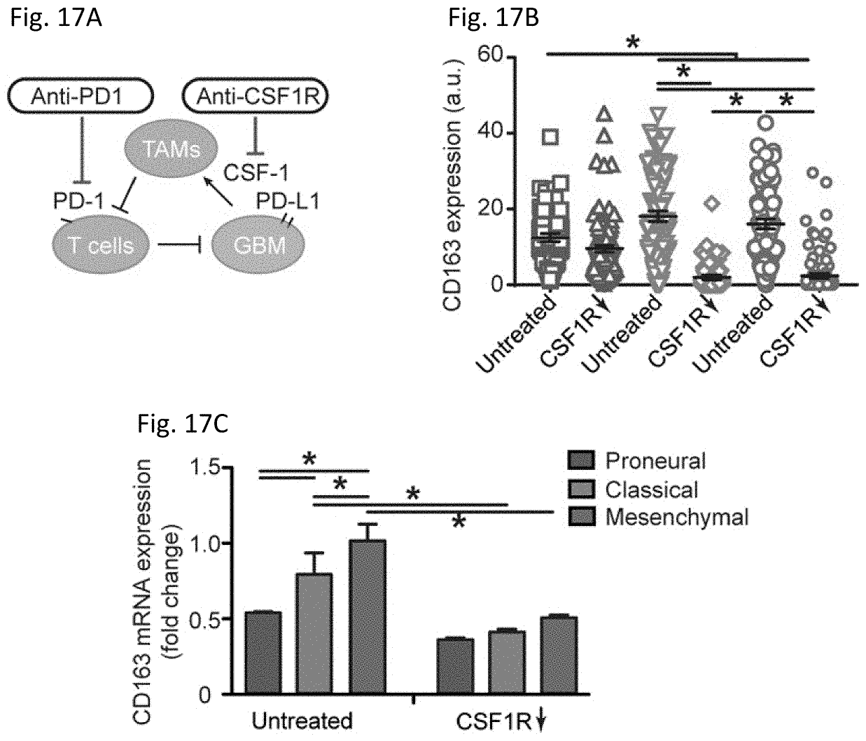

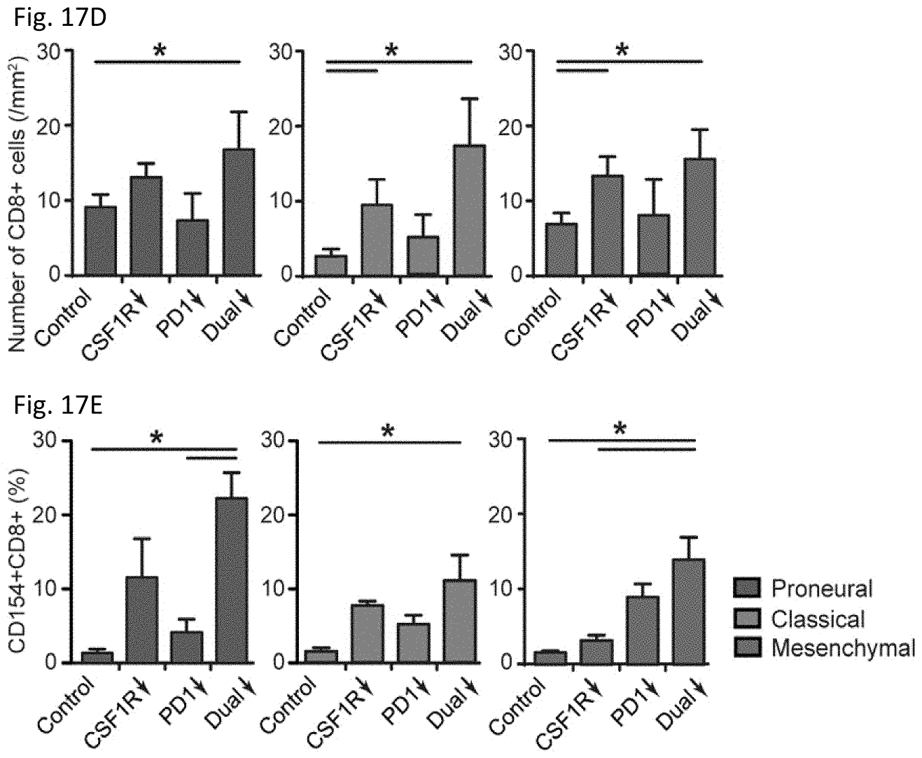

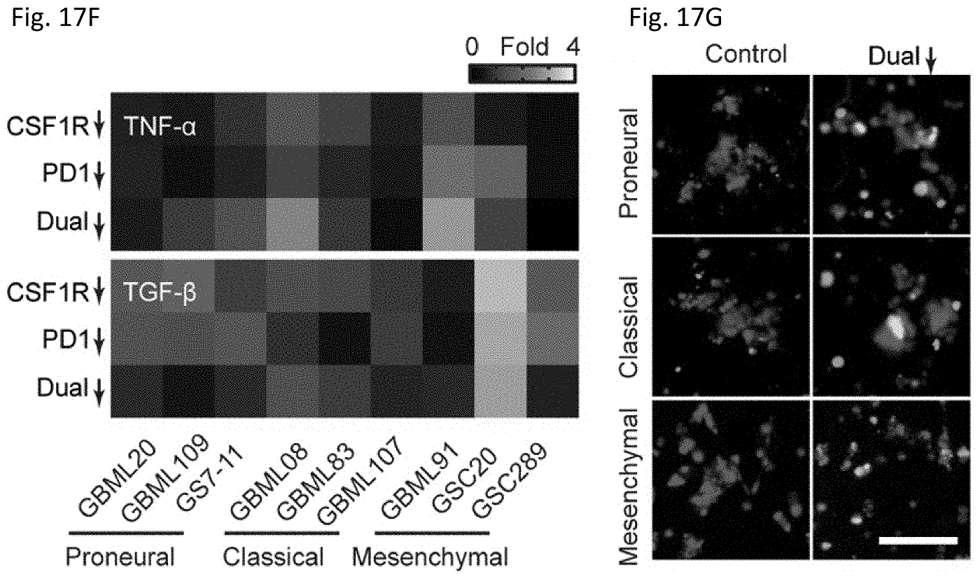

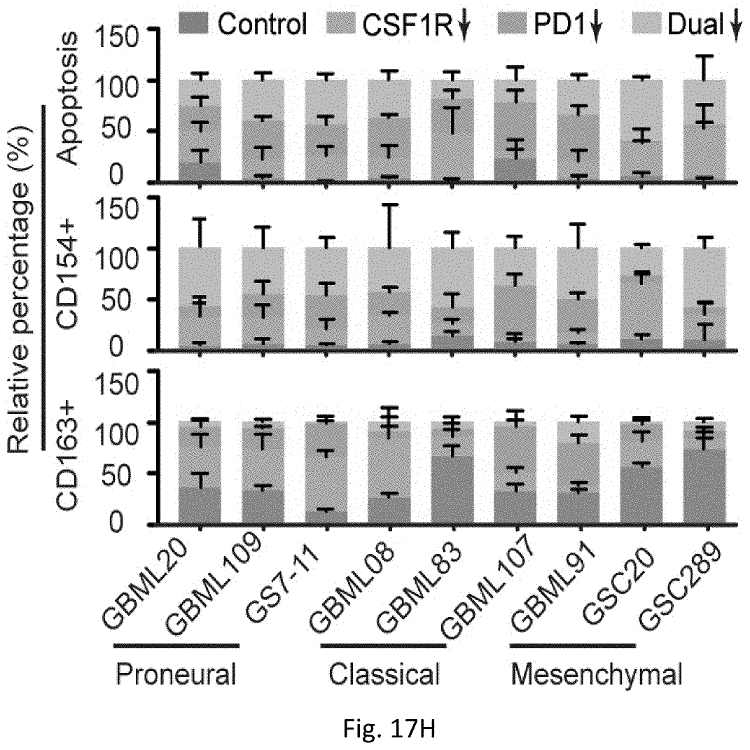

Cytokine concentrations in supernatants were quantified by using human IL-10 (430604, BioLegend), TGF-β1 (88-8350-22, Invitrogen), IFN-γ (430104, BioLegend), and TNF-α (430204, BioLegend) ELISA kits, respectively, according to manufacturer's protocol after collecting supernatants and centrifuging at 2000×g for 10 min at 4° C. to remove cellular debris. Data was normalized for each type of cytokine using STANDARDIZE function in Excel for FIG. 10A through FIG. 10G and normalized to control in FIG. 17A through FIG. 17G.

Inhibition Assays

CSF-1R inhibitor BLZ945 (0.1 μg/ml) and PD-1 blockade nivolumab (1 μg/ml) were used in the monotherapy or dual inhibition treatments. Control groups were treated with fresh cell culture media supplemented with DMSO (0.01%) and human IgG4 isotype control antibody (1 μg/mL, BioLegend). Fresh cell culture media supplemented anti-PD-1 and anti-CSF-1R antibodies was loaded in the microfluidic channels 2 hr after loading allogeneic CD8+ T-cells. Blocking media was freshly prepared and replenished every 24 hr for 3 days.

Adhesion Assay of TAM on HBMVEC Capillary

TAMs were obtained by co-culture macrophages with different GBM subtypes using transwell for 3 days. The GBM-educated TAMs were retrieved from transwell and labeled with CellTracker Green (10 μM, C2925, Thermo Fisher-Scientific). HBMVECs were labeled with CellTracker Red (10 μM, C34552, Thermo Fisher-Scientific), and seeded at 100,000 cells/well onto Matrigel pre-coated 24 well-plate (200 μL/well at 37° C. for 30 min), and cultured 12 hr to allow for capillary formation. Following the HBMVEC capillary formation, the pre-labeled TAMs were seeded at 100,000 cells/well into the 24 well-plate. Following a 12 hr incubation at 37° C., the unattached TAMs were washed away with warm HBMVEC media for three times. The attached TAMs on HBMVEC capillary were imaged with 20× objective. The adhered TAMs were then manually counted and plotted as cell number per 10⁴ μm² HBMVEC area.

GBM ECM Composition Analysis

After GBM cells (L08, L20, or L09) and TAMs were cultured in the brain tissue-mimicking hydrogel for 1 and 3 days, different ECM components were fixed and stained with Dylight 488-Laminin (PA522901, Thermo Fisher Scientific), PE-Fibronectin (IC1918P, R and D Systems), and APC-Collagen IV (51-9871-80, Thermo Fisher Scientific) per vendors' instructions. To characterize the HA accumulation, the devices were incubated with Biotinylated Hyaluronic Acid Binding Protein (385911-50 UG, Millipore Sigma) followed by staining with Streptavidin, Alexa Fluor 647 conjugate (S21374, Thermo Fisher Scientific). Each fluorescently stained device was imaged with 6-10 random images using a fluorescent microscope (Zeiss Axio Observer.Z1) with a 40× objective. The total fluorescent intensity of each image field was quantified using ImageJ (NIH), normalized to its respective DAPI intensity and compared between groups.

Flow Cytometry

To quantify the purity of PBMC-isolated CD8+ cell, cells were washed twice with cold cell sorting buffer containing 2 mM EDTA and 1% BSA in PBS on ice for 10 min. Then, fluorochrome-conjugated antibodies for CD8 (Alexa Fluor 647 anti-human CD8, 344726, BioLegend) were used to label cells for 30 min at 4° C. After washing with 5% BSA buffer solution in PBS twice, labeled cells were measured with a LSRII analyzer (BD Biosciences). All data were analyzed using FlowJo software (Tree Star Inc).

Confocal Microscopy

3D Z-stack images of the engineered GBM microfluidic model were acquired with a Zeiss LSM 710 Laser Scanning Confocal Microscopes with a 20× objective lens (N.A.=0.4). To visualize different cell comparts in the model, HBMVEC, GBM cell, and CD8+ T-cell were labeled with DiD (5 μM, V22887, Thermo Fisher-Scientific), CellTracker Red (5 μM, C34552, Thermo Fisher-Scientific) and CellTracker Green (5 μM, C2925, Thermo Fisher-Scientific), respectively prior to loading cells in microfluidics model. All stacked images were reconstructed by using ZEN lite software (Zeiss) for 3D visualization.

qPCR Analysis

RNA was extracted using RNeasy Plus Mini Kit (Qiagen, US), and cDNA was synthesized from the mRNA using SuperScript IV VILO Master Mix for RT-RCR (Invitrogen, US), followed by real-time PCR using a SYBR Green PCR Master Mix (Thermo Fisher Scientific, US) and QuantStudio six sequence detection system (Applied Biosystems, Thermo Fisher Scientific). The reactions were performed with the following cycling conditions: 95° C. for 10 min followed by 40 cycles of 95° C. for 15 s and 60° C. for 1 min. GAPDH was used as a housekeeping gene for normalization. All experiments were repeated three times. The relative change in gene expression was analyzed with the 2^{-ΔΔCT} method. The used primers are listed below: CD154 (Forward: CTGATGAAGGGACTTGAC (SEQ ID NO. 2), Reverse: TCTACAGCTTGAACATGC (SEQ ID NO. 3)), CD163 (Forward: 5′-CAGGAAACCAGTCCCAAACA-3′(SEQ ID NO. 4), Reverse: 5′-AGCGACCTCCTCCATTTACC-3′ (SEQ ID NO. 5)), PD-1 (Forward: CGTGGCCTATCCACTCCTCA (SEQ ID NO. 6), Reverse: ATCCCTTGTCCCAGCCACTC (SEQ ID NO. 7)), PD-L1 (Forward: 5′-AAATGGAACCTGGCGAAAGC-3′ (SEQ ID NO. 8), Reverse: 5′-GATGAGCCCCTCAGGCATTT-3′ (SEQ ID NO. 9)), CSF1 (Forward: GCTGTTGTTGGTCTGTCTC (SEQ ID NO. 10), Reverse: CATGCTCTTCATAATCCTTG (SEQ ID NO. 11)), GAPDH (Forward: 5′-GAGTCAACGGATTTGGTCGT-3′ (SEQ ID NO. 12), Reverse: 5′-TTGATTTTGGAGGGATCTCG-3′ (SEQ ID NO. 13)).

DNA Methylation and Data Analysis

To assess the epigenetic modifications of TAM in molecularly distinct GBMs, GBM cells and TAMs were co-cultured in the brain tissue-mimicking hydrogel for 3 days, and then recovered using Cell Recovery solution (Corning, N.Y.). TAMs pre-labeled with CellTracker Green (10 μM, C2925, Thermo Fisher-Scientific) and GBM cells were then isolated by using a flow cytometry (MoFlo XDP cell sorter, Beckman Coulter) for DNA methylation. DNA was extracted using automated Maxwell Promega protocol. Whole Genome DNA methylation profiling was performed using Illumina EPIC array, as described previously (Serrano J et al., InGlioblastoma 2018 (pp. 31-51). Humana Press, New York, N.Y.). DNA methylation profiling was performed on all the cases and the raw idats generated from iScan were processed and analyzed using Bioconductor R package Minfi (Aryee M J et al., Bioinformatics. 2014 May 15; 30(10):1363-9). All the Illumina array probes were normalized using quantile normalization and corrected for background signal. Samples were then checked for their quality using mean detection p-values and probes with mean detection p-value<0.05 were used for further downstream analysis. Beta values were obtained from the probes that passed the QC as mentioned above. To identify the differentially methylated CpG probes between different groups, dmpFinder function from Minfi package was used. Probes with FDR cutoff (q<0.05) were considered as most significantly variable probes. Beta value<0.2 means Hypomethylation and Beta value >0.8 means Hypermethylation. For all the differentially methylated CpGs, clustering of samples was analyzed using t-distributed stochastic neighbor embedding (TSNE) method (Van der Maaten L et al.,Journal of machine learning research. 2008 Nov. 1; 9 (11)) that was applied on the 10,000 most variable probes obtained using Minfi package. Heatmaps were generated in a semi-supervised manner using pheatmap package in R, which shows the hierarchical clustering pattern of the top 10,000 significant differentially methylated probes across patients.

KEGG Pathway Analysis

For finding the most enriched signaling pathways, the most significantly variable probes/genes across groups were taken and passed through ClusterProfiler (Yu G et al., Omics: a journal of integrative biology. 2012 May 1; 16(5):284-7) R package for KEGG (Kyoto Encyclopedia of Genes and Genomes) enrichment. The dot plots represent ratio of genes (x-axis) involved in each signaling pathway (y-axis) of KEGG database (Kanehisa M et al., Nucleic acids research. 2000 Jan. 1; 28(1):27-30). Size of the dots shows the gene counts and the color denotes the significance level.

MethylCIBERSORT for Immune Cell Population Calculation

To calculate the amount of immune cell population in the cases, MethylCIBERSORT (Chakravarthy A et al., Nature communications. 2018 Aug. 13; 9(1):1-3) was used, which is a deconvolution R package used to accurately estimate the cellular composition and tumor purity from DNA methylation data. Beta values obtained from raw idats along with the signature genes were passed through the CIBERSORT (as mentioned in the paper) to deconvolute the immune cell population in the cases.

Statistical Analysis

All data were from at least three independent experiments, and presented as means±s.e.m (standard error of the mean). The means of groups were compared using one-way analysis of variance (ANOVA) followed by Tukey's post-hoc test in GraphPad Prism or unpaired, two-tailed Student's t-test in Excel (Microsoft), as shown in figure captions. p-Value smaller than 0.05 was considered statistically significant.

The results are now described.

Clinicopathological Markers Fail to Predict PD-1 Response

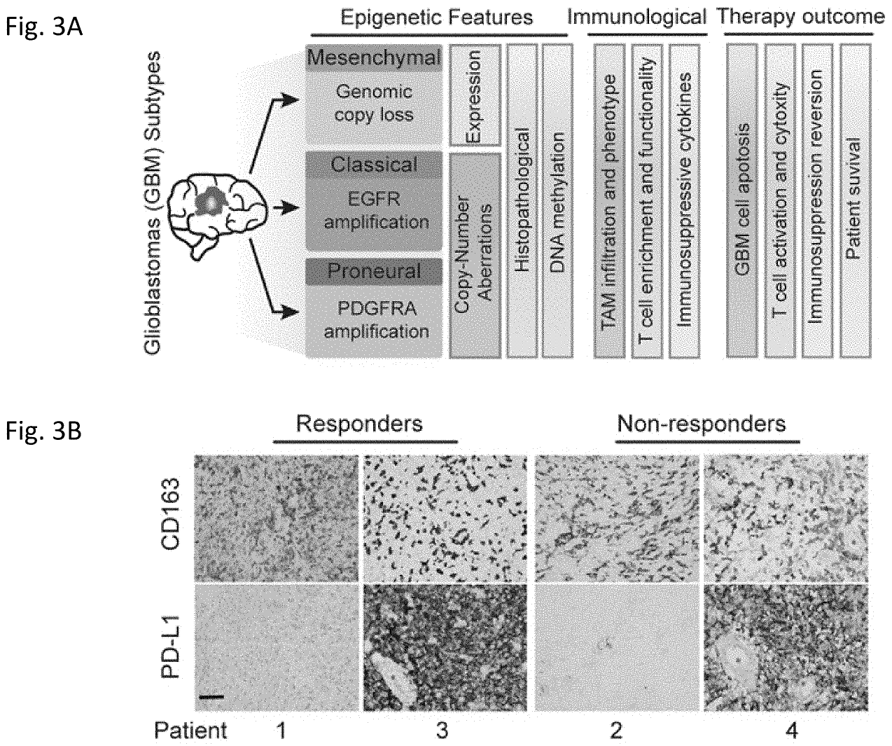

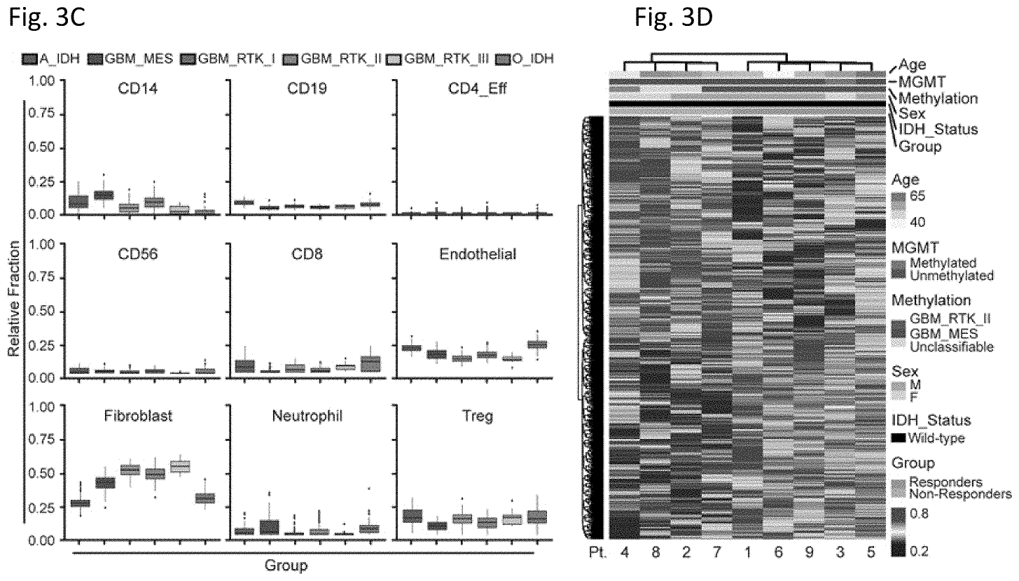

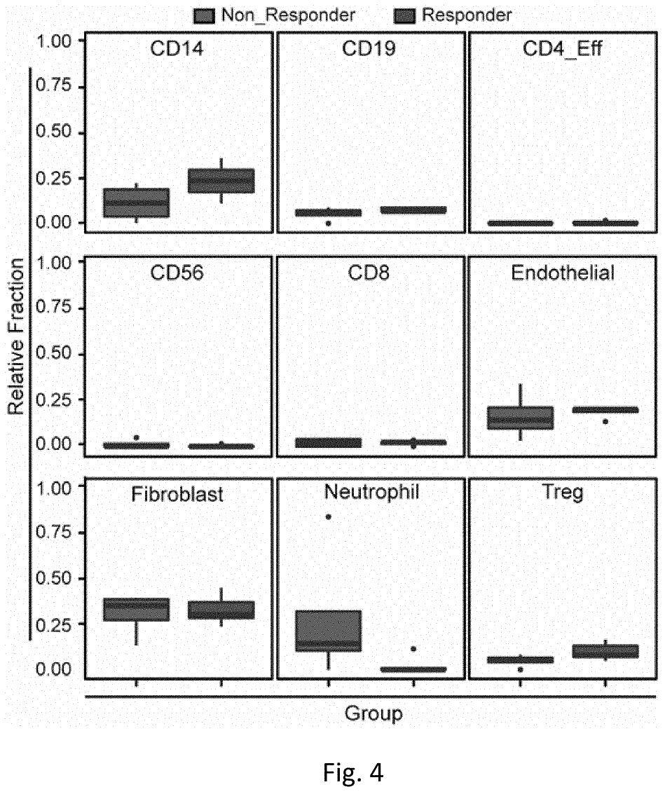

To explore the heterogeneity of the immunosuppressive GBM microenvironments, a cohort of IDH-wildtype GBM tumors (FIG. 3A) from patients treated with PD-1 inhibitor (nivolumab) were analyzed for 2-15 months (median 3.7 months). All primary tumors were classified by clinically validated and New York State approved whole genome DNA methylation classification (Capper D et al., Nature. 2018 March; 555(7697):469-74), MGMT promoter methylation, RNA expression, DNA Copy-Number and next-generation sequencing mutation analysis (FIG. 4; Bayin N S et al., Neoplasia. 2016 Dec. 1; 18(12):795-805). Diagnostic samples were analyzed for PD-L1 and CD163 expressions by immunohistochemistry. Clinical data indicated that PD-L1 staining was not predictive of response, showing both strong or no expression in both responders and non-responders across different GBM subtypes (FIG. 3B). Meanwhile, all tumors showed marked TAM infiltration by CD163, irrespective of molecular subtype (FIG. 3B). The aggressive TAM infiltration present in the perivascular, infiltrative and tumor bulk regions was concurrent with GBM tumor progression, indicating TAM-GBM tumor interactions contribute to the immunosuppressive GBM microenvironments and therapy resistance. GBM methylCIBERSORT analysis of a cohort of 435 glioma samples previously profiled (Capper D et al., Nature. 2018 March; 555(7697):469-74) further revealed prominent CD14 monocytic and neutrophilic DNA methylation and low CD8+ T-cell methylation signatures in MES patients, and low CD14 monocytic and neutrophilic DNA methylation and high CD8+ T-cell methylation in PN (RTK_I) patients (FIG. 3C). However, there was no significant difference between responders and non-responders in DNA methylation in these immune cell signatures (FIG. 4). In addition, clinicopathological methylation analysis also revealed diverse immunosuppressive signatures in distinct GBM subtypes, and some differences in epigenetic signatures between responders and non-responders (FIG. 3D), but these were insufficient for predicting the response. These data together confirm that current static biomarkers seem to be poor predictors of anti-PD-1 immunotherapy response, and a further analysis of the heterogeneity of the immunosuppressive GBM microenvironments might help identify niche-associated mechanisms for predicting and improving patient-specific responses to immunotherapy.

Modeling the GBM Tumor Niche in an Ex Vivo ‘GBM-On-a-Chip’ Microphysiological System

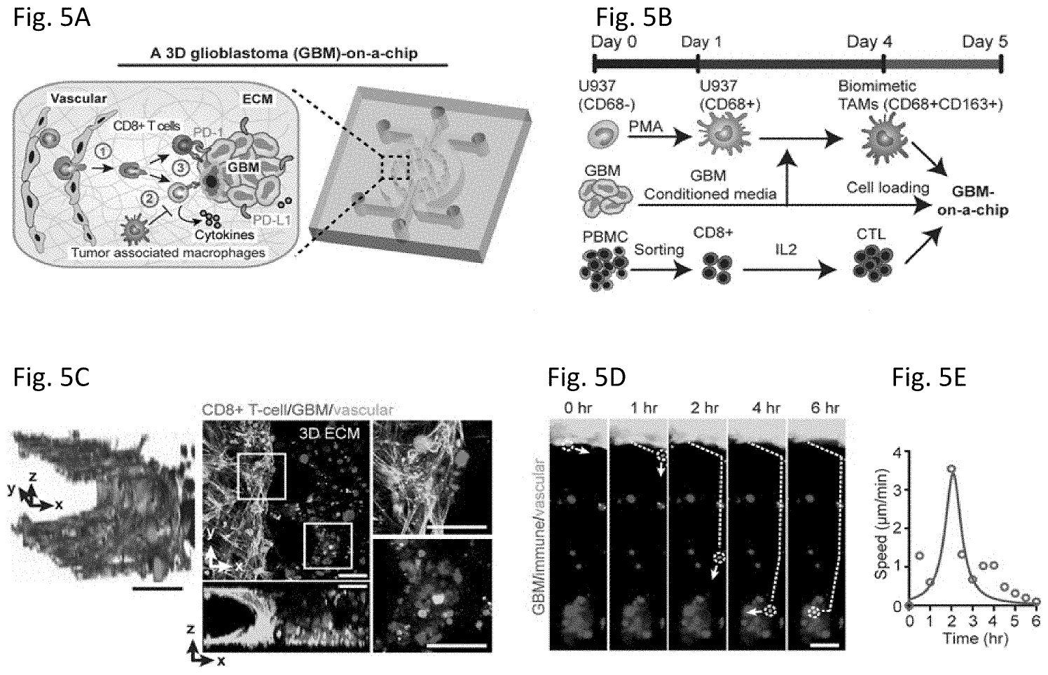

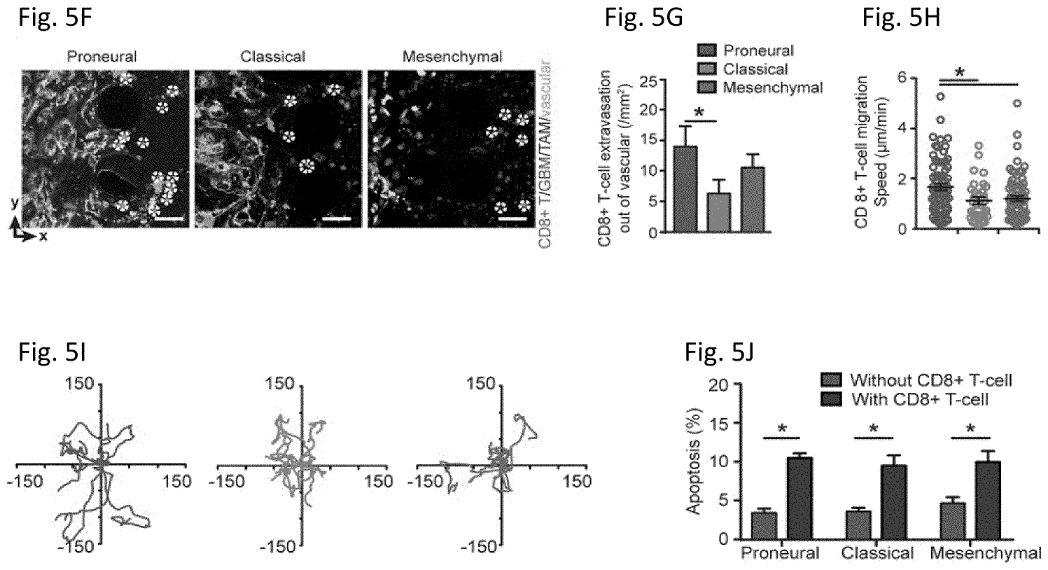

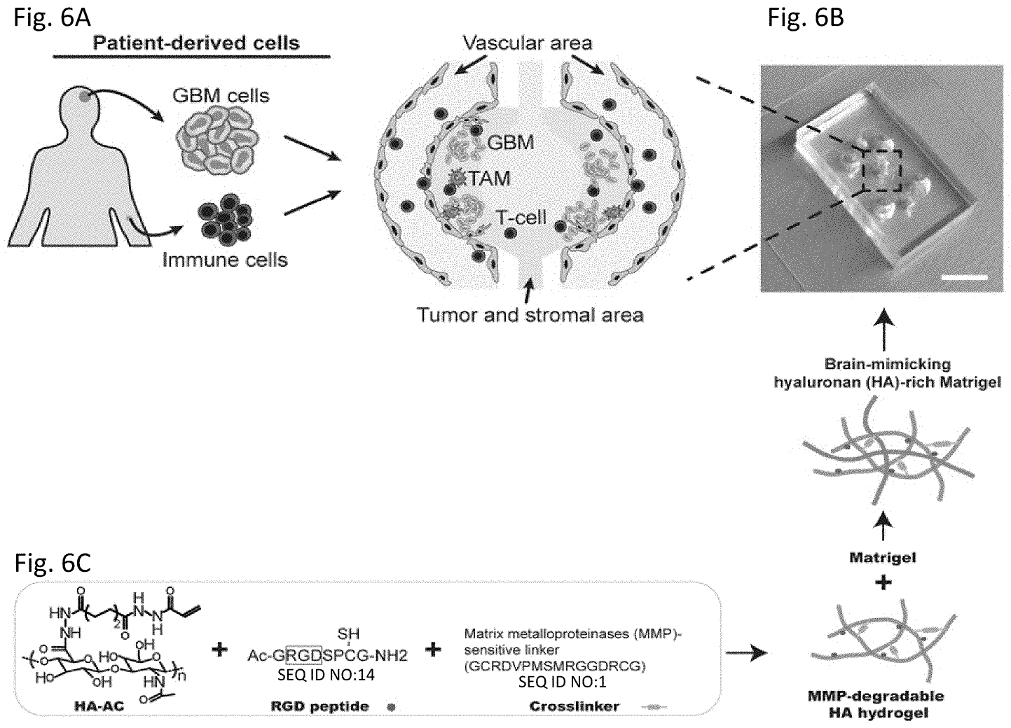

To dissect the heterogeneity of anti-PD-1 immunotherapy responses in molecularly distinct GBM cohort of PN, CL, and MES subtypes, a microfluidics-based 3D ‘GBM-on-a-Chip’ microphysiological system was developed (FIG. 5A through FIG. 5J; FIG. 6A through FIG. 6C), mimicking the subtype-specific in vivo GBM tumor niche. This organotypic system housed a 3D brain microvessel (FIG. 5A through FIG. 5C, yellow) derived from human brain microvascular endothelial cells (hBMVECs), TAMs derived from human macrophages (FIG. 6A and FIG. 6B), patient-derived and molecularly-distinct GBM cells (FIG. 5A through FIG. 5C, red), and sorted allogeneic human CD8+ T-cells (FIG. 5A through FIG. 5C, green) from primary peripheral blood mononuclear cells (PBMCs) within a 3D brain-mimicking hyaluronan (HA)-rich Matrigel extracellular matrix (ECM) (FIG. 7A through FIG. 7D; Wang H et al., Advanced healthcare materials. 2019 February; 8(4):1801234). Specifically, ‘GBM-on-a-Chip’ culture was compartmentalized by a peripheral channel designated for patterning 3D brain microvessels (outer ring), an intermediate tumor stromal area (middle ring), and a core media region (center region) for long-term media supply (FIG. 5A; FIG. 6A through FIG. 6C). The three compartments were segregated by regularly spaced micropillars that confine cell-embedded hydrogels to mimic the native in vivo pathological architecture of GBM tumors. To reconstitute in vivo GBM tumor composition in vitro, biomimetic human TAMs were nested within the 3D engineered HA-rich ECM tissue (FIG. 5F, blue), making up 30% cell volume (Hambardzumyan D et al., Nature neuroscience. 2016 January; 19(1):20). TAMs were differentiated from U937 monocytes with Phorbol 12-myristate 13-acetate (PMA) and treated with conditioned media from patient-derived adult GBM cells of all three major molecular subtypes (FIG. 5B; Shi Y et al., Nature communications. 2017 Jun. 1; 8(1):1-7). To mimic in vivo extravasation events of adaptive immune responses of CTLs for different GBM patients, IL-2-activated allogeneic human CD8+ T-cells were circulated into the 3D brain microvessel and their extravasation dynamics were profiled as they migrated through brain vasculature, interacted with TAMs, and interacted with GBM tumor cells (FIG. 5D through FIG. 5J).

Distinct Extravasation and Cytotoxic Activities of Allogeneic CD8+ T-Cells in GBM Subtypes

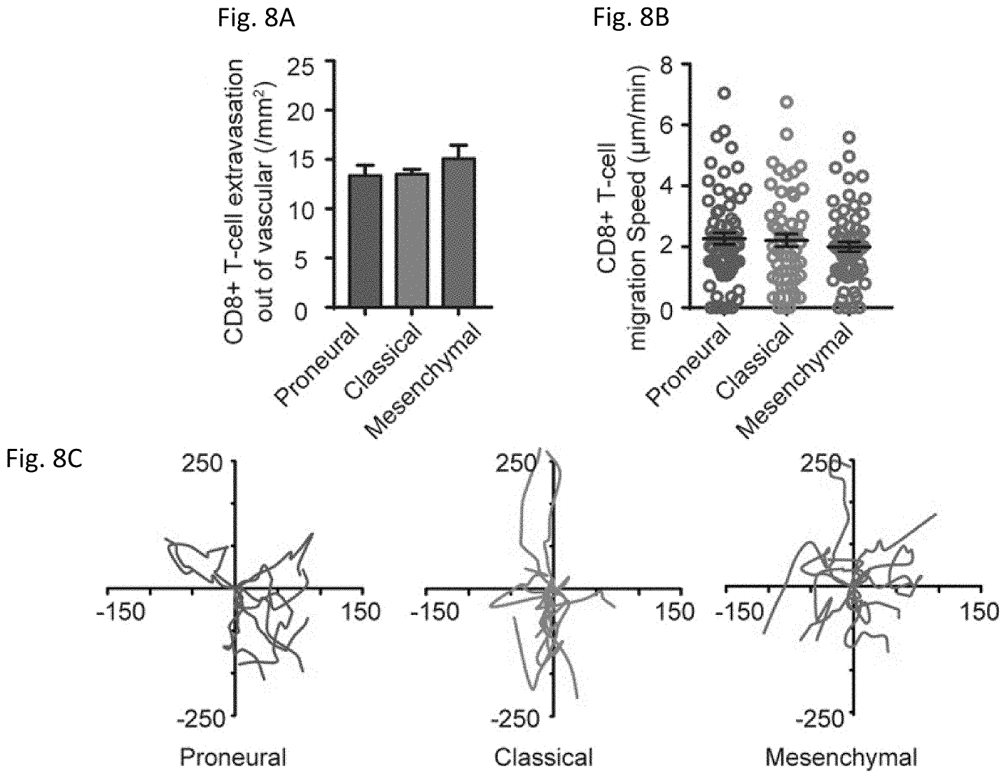

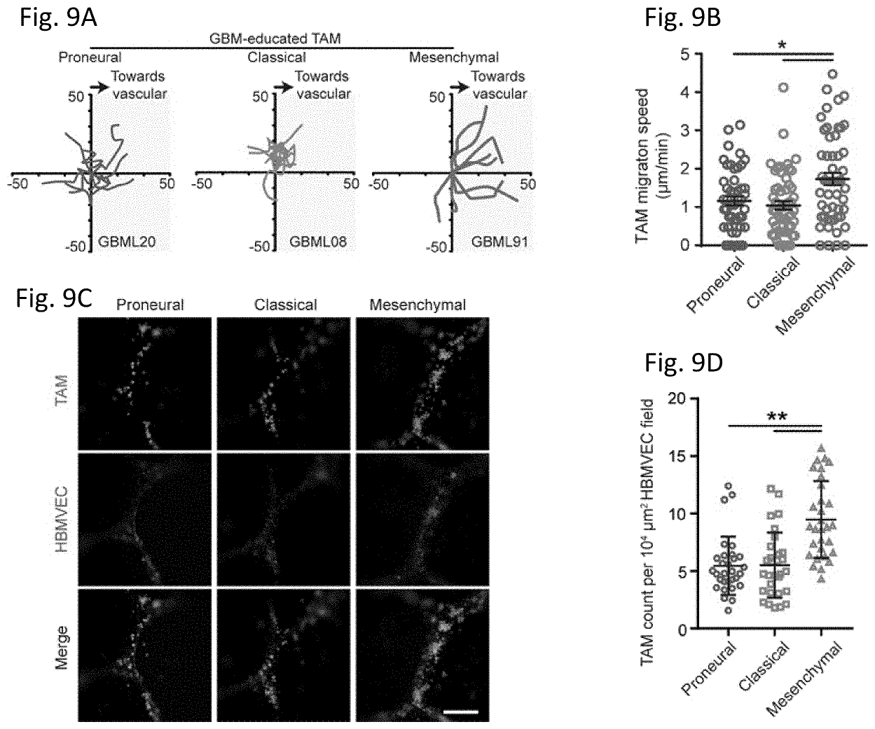

The dynamic extravasation, migration, and cytotoxic activities of primary allogeneic human CD8+ T-cells in the engineered GBM niches were charted in real-time to mechanistically understand CTL activity across molecularly distinct GBMs. Under time-lapsed imaging (FIG. 5D), a single CD8+ T-cell's extravasation in three stages was monitored on ‘GBM-on-a-Chip’: transmigration (0-1 hr) through the patterned brain microvessel, penetration (1-4 hr) into the brain-mimicking tissue construct, and interactions with GBM tumors (4-6 hr) at a single-cell level (FIG. 5E). The number of allogeneic CD8+ T-cell extravasation was quantified in molecularly distinct GBMs (FIG. 5F and FIG. 5G), cell migration trajectories (FIG. 5H) and migration speed (FIG. 5I). PN (GBML20) GBM was demonstrated to exhibit significant increases both in the number and speed of allogeneic CD8+ T-cell infiltrate compared to the CL (GBML08) and MES (GBML91) GBM samples after 3 days' culture (FIG. 5G through FIG. 5I), which is consistent with clinical observations (FIG. 3C). Moreover, stark differences were observed in T-cell migration trajectories, where the PN GBM demonstrated free, active motion of CD8+ T-cell while the degree of CD8+ T-cell motility was limited in the CL GBM and, arrested in the MES GBM (FIG. 5H). Interestingly, in the absence of TAM, increased number of allogeneic CD8+ T-cell extravasation (15 cell/mm²) was observed, suggesting that the presence of TAM inhibits CD8+ T-cell extravasation (FIG. 8A through FIG. 8C). The data also showed that the MES GBM-educated TAM exhibited faster motion toward the brain microvessels, relative to the PN GBM-educated TAM (FIG. 9A through FIG. 9D).

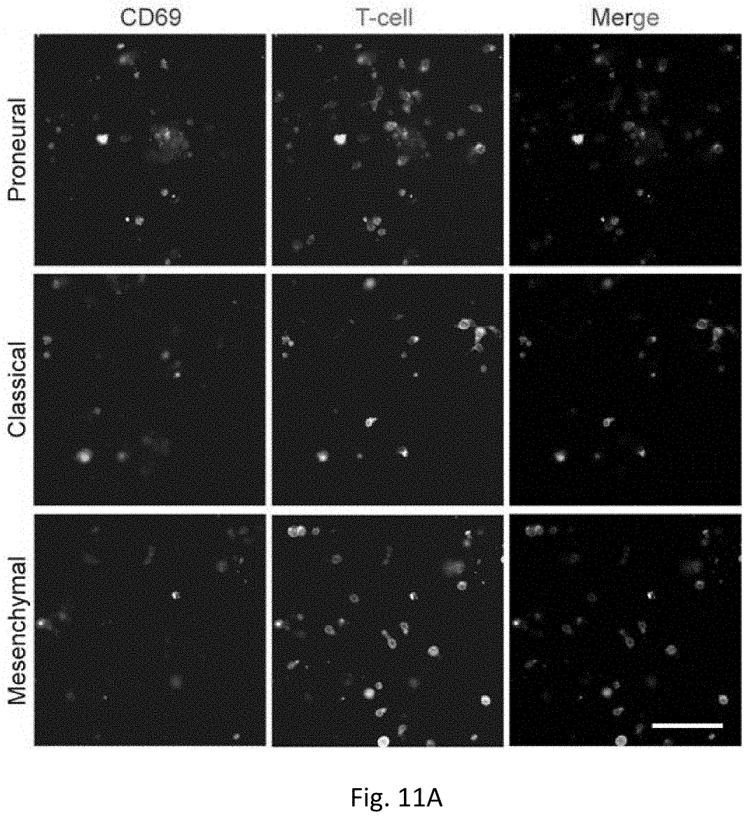

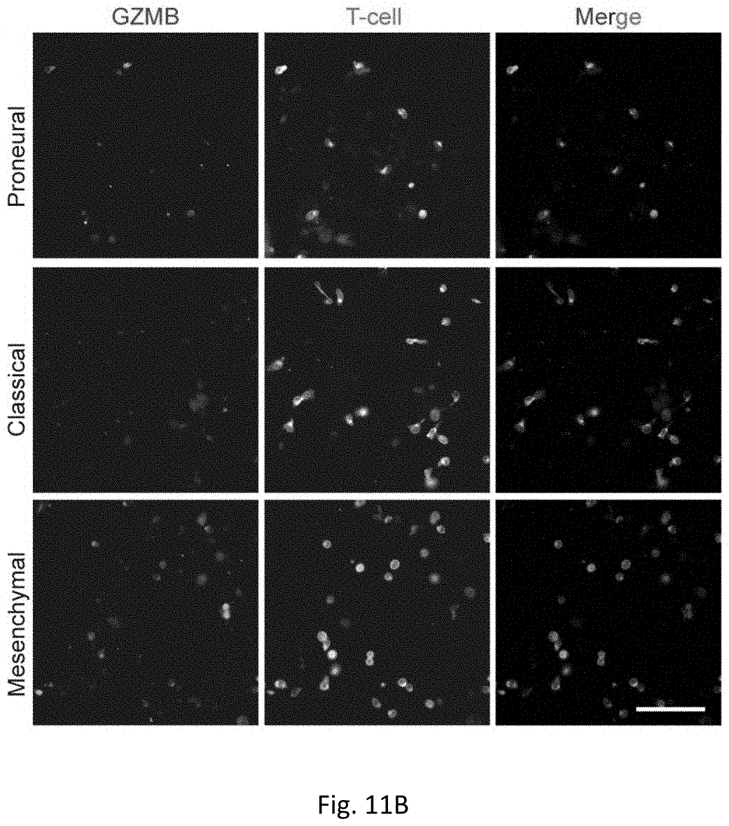

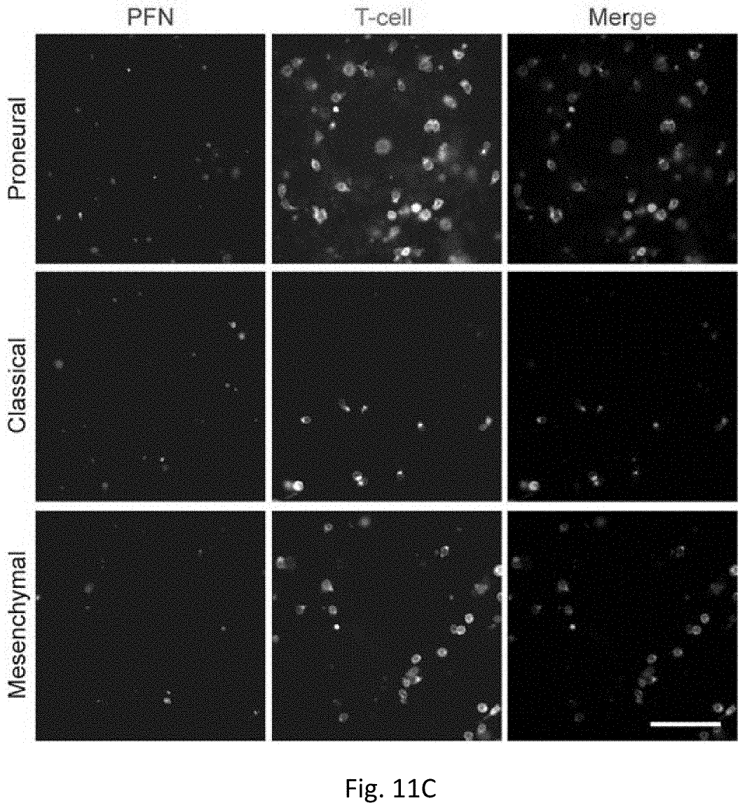

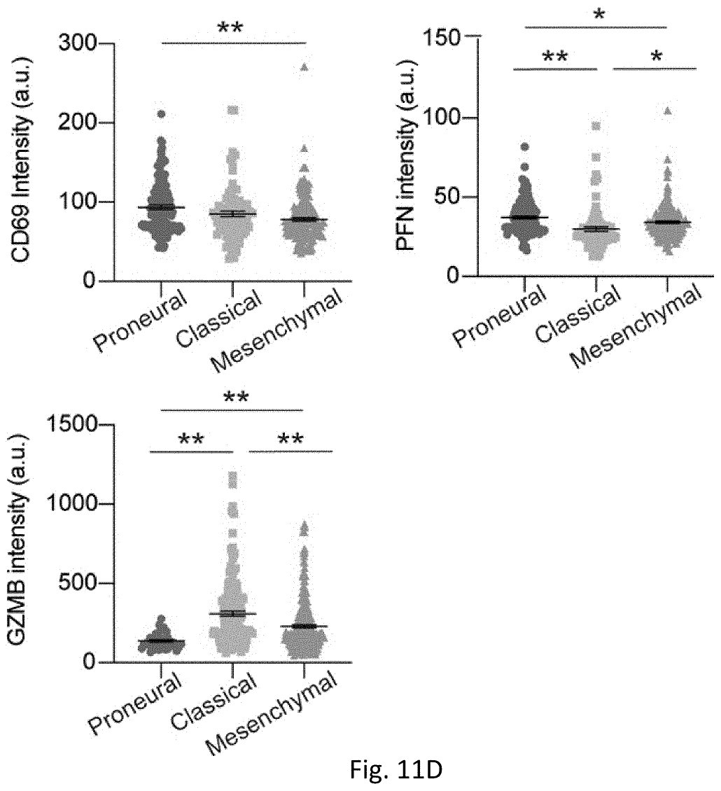

The cytotoxic activities of IL-2-activated allogeneic CD8+ T-cell on different subtypes of GBM cells were confirmed with a higher apoptosis ratios of GBM cells as compared to that of without allogeneic CD8+ T-cell in the niche (FIG. 5J). To further understand the cytotoxic function of the allogeneic CD8+ effector T-cell, the T-cell in different GBM niches were stained with T-cell activation markers CD154 and CD69, and cytotoxic function markers such as granzyme B (GZMB) and perforin (PFN) (FIG. 10A through FIG. 10G; FIG. 11A through FIG. 11D). The on-chip staining showed that most of IL-2-activated allogeneic CD8+ T-cells expressed GZMB and CD69 but weak PFN after cultured in the GBM niches, while CD8+ T-cell in the MES GBM niche overall had lower expressions of these T-cell activation and cytotoxic function markers (FIG. 11A through FIG. 11D), demonstrating the immunosuppressive feature of the MES GBM niche. Meanwhile, both immunostaining (FIG. 10B) and qPCR analysis (FIG. 10C) showed that the ratio of activated CD154+CD8+ T-cells were markedly decreased in all GBM subtypes, while more significantly in the MES GBM niche, when compared to CD8+ T-cells cultured without the GBM niche. It confirmed that the immunosuppressive milieu hindered CTL activation and cytotoxic function at different levels of severity in molecularly distinct GBM subtypes.

GBM Subtypes Differentially Regulate TAM Phenotype, Epigenetics, and Immunity

The phenotypic status of cytotoxic CD8+ T-cell, TAM, and GBM cell across GBM molecular subtypes was next determined (FIG. 10A). Off-chip cell staining was performed after on-chip cell recovery with specific cell surface markers for CD8+ T-cell activation (CD154+), immune checkpoints (PD-1 and PD-L1) and macrophage phenotype [CD68 for identifying macrophage and CD163 for anti-inflammatory ‘M2’-like TAM (Lu-Emerson C et al., Neuro-oncology. 2013 Aug. 1; 15(8):1079-87)]. As PD-1 expression is a marker of T-cell activation, the PD-1 expressions on different GBM-activated allogeneic CD8+ T-cells were compared and normalized to the baseline value of PD-1 expression on CD8+ T-cell without tumor activation with immunostaining (FIG. 10B) and qPCR analysis (FIG. 10C). The results confirmed that while PD-1 expression was low on untreated PBMC-derived CD8+ T-cell, it was elevated on the tumor-activated PBMC-derived CD8+ T-cells in most GBM niches. Particularly, the MES GBM tumor niche was characterized with highest expressions of PD-1 on CD8+ T-cell and PD-L1 on patient-derived GBM cell (FIG. 10B). Further qPCR analysis confirmed the strong levels of PD-1 expression in MES GBM treated CD8+ T-cell and PD-L1 in MES GBM cell (FIG. 10D). The PD-L1 mRNA expressions in GBM cell (FIG. 10C) and TAM (FIG. 11A through FIG. 11D) varied across different GBM subtypes. Since the majority of intra-tumoral immune cells in GBM were represented by TAM (FIG. 3B and FIG. 3C), the immunosuppressive TAM activity was analyzed in GBM-on-a-Chip. Both immunostaining (FIG. 10D through FIG. 10E) and qPCR analysis (FIG. 17C) results showed a significant number of immunosuppressive CD163+M2-TAMs in the MES GBM compared to that in the PN and CL subtypes, which is consistent with patient sample immunohistochemistry and methylCIBERSORT data (FIG. 3A through FIG. 3D).

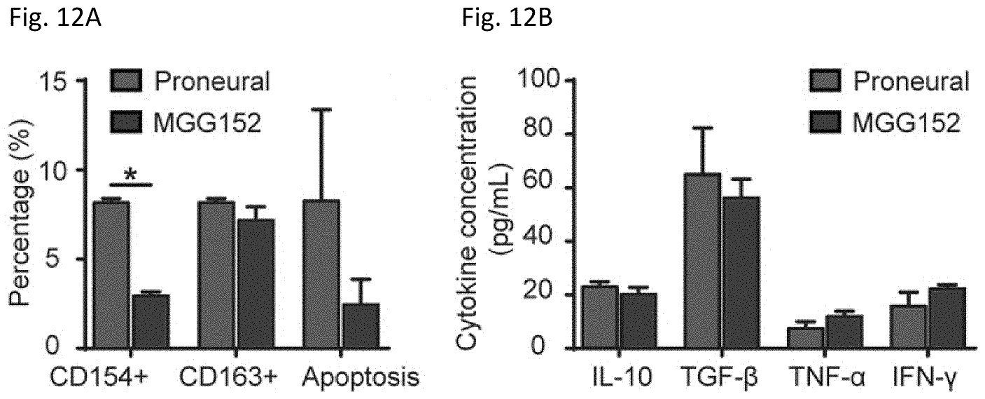

The anti- and pro-inflammatory cytokines were mapped by using enzyme-linked immunosorbent assay (ELISA) in nine patient-derived GBM lines to further analyze the properties of the immunosuppressive cytokine milieus across GBM patient subtypes. CSF-1 has been shown to influence macrophage polarization toward a M2 phenotype in GBM (Pyonteck S M et al., Nature medicine. 2013 October; 19(10):1264); however, it is unclear if molecularly different GBM subtypes have distinct CSF-1 secretion profiles. CSF-1 was highly secreted in the MES GBM, compared to the PN and CL subtypes using qPCR analysis (FIG. 10C) and ELISA assay (FIG. 10F). These results thus demonstrate CSF-1 signaling as an ideal therapeutic target in all GBM subtypes (Zhu Y et al., Cancer research. 2014 Sep. 15; 74(18):5057-69). Furthermore, the results showed that different GBM patient-derived cells showed distinct immunosuppressive cytokine milieus, and MES and CL GBMs likely had higher productions of anti-inflammatory cytokines TGF-β1 and IL-10, compared to the pro-inflammatory cytokines TNF-α and IFN-γ (FIG. 10G), driving TAM polarization toward a M2-like phenotype. IDH-wildtype GBMs have been reported to display a greater number of tumor-infiltrating lymphocytes and elevated PD-L1 expression compared to IDH-mutant GBMs (Berghoff A S et al., Neuro-oncology. 2017 Oct. 19; 19(11):1460-8), thus IDH mutational status may contribute differently to adaptive immune responses. However, the IDH-mutant GBM (patient MGG152) showed similar cellular and cytokine conditions to the IDH-wildtype PN GBM in the on-chip study, which may be due to the poor survival capability of these IDH-mutant GBM cells when cultured in vitro (FIG. 12A and FIG. 12B).

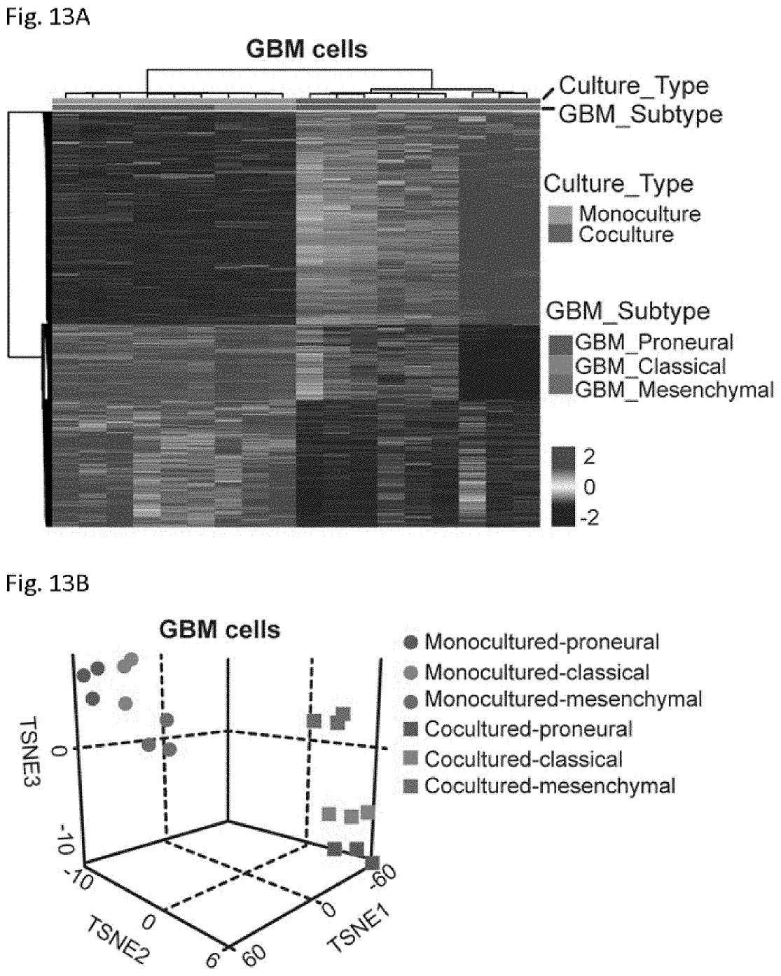

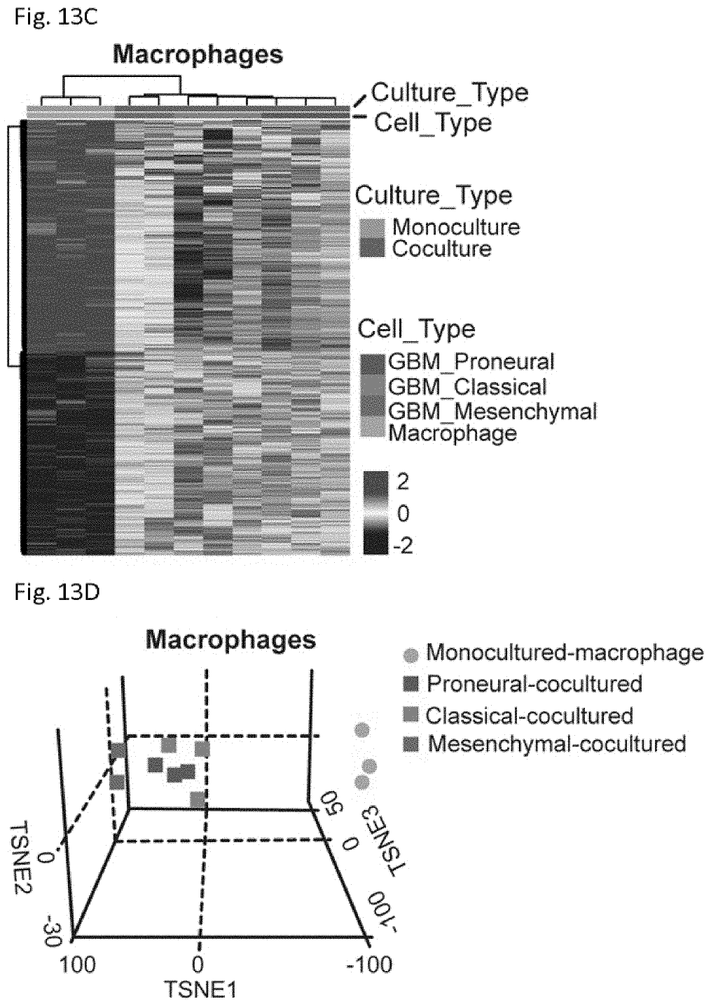

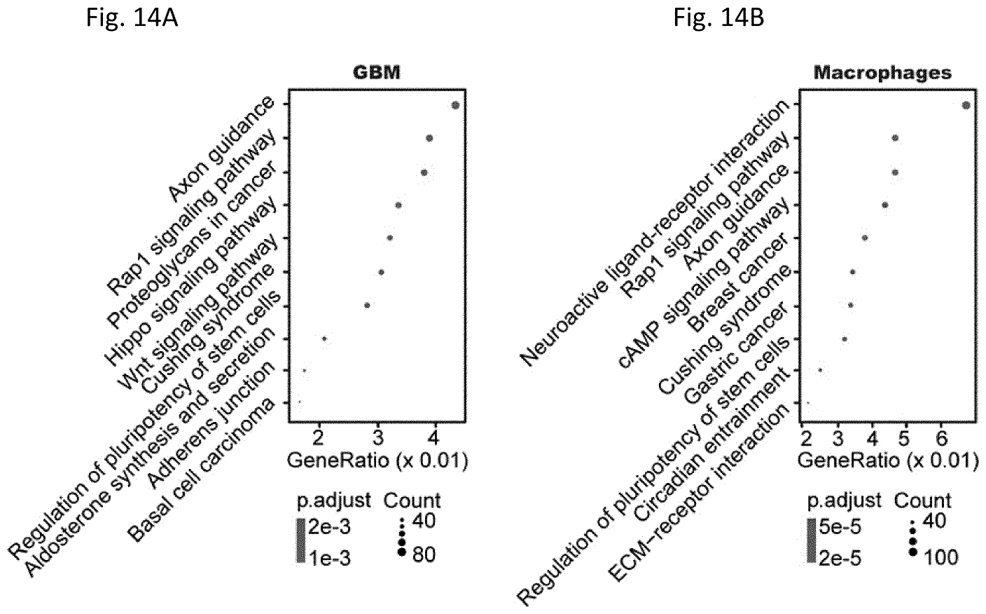

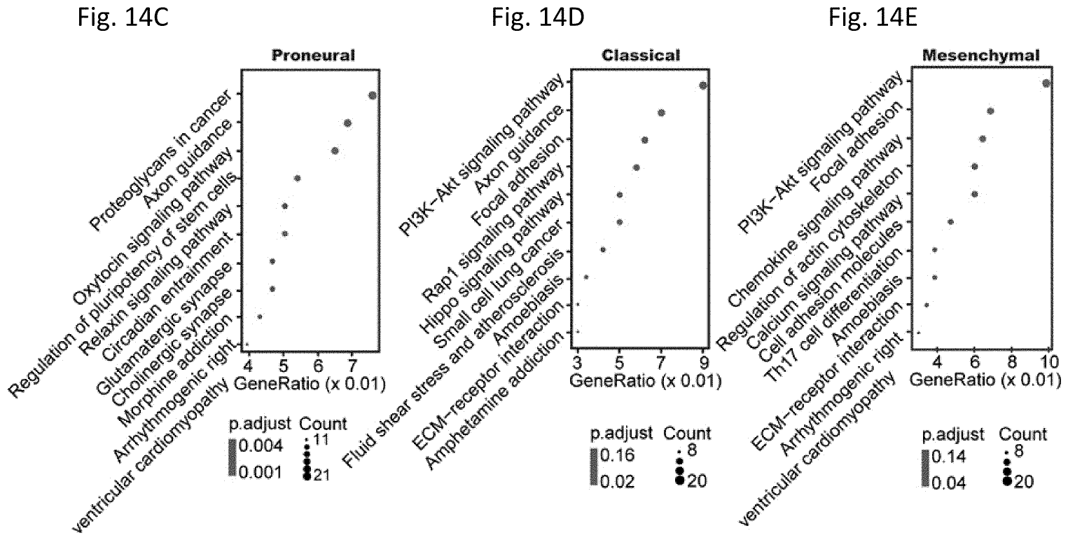

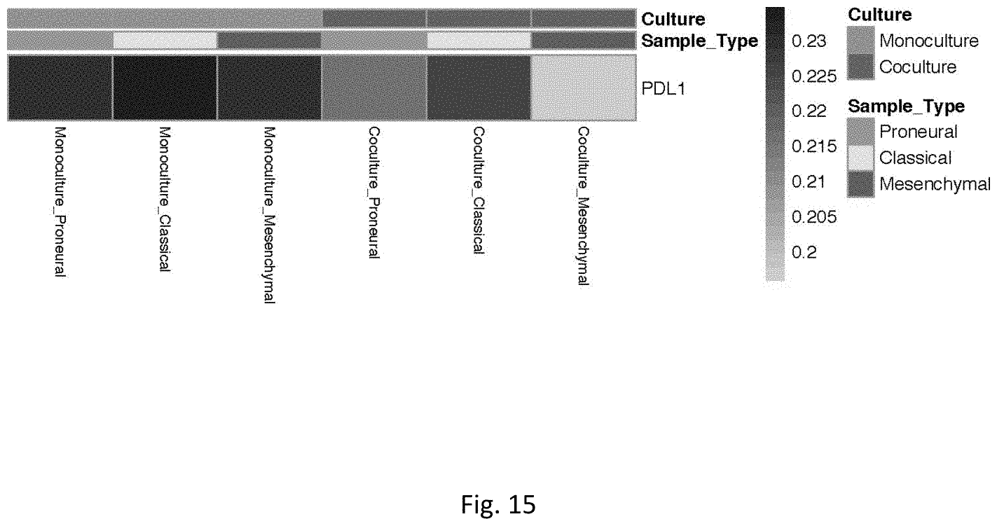

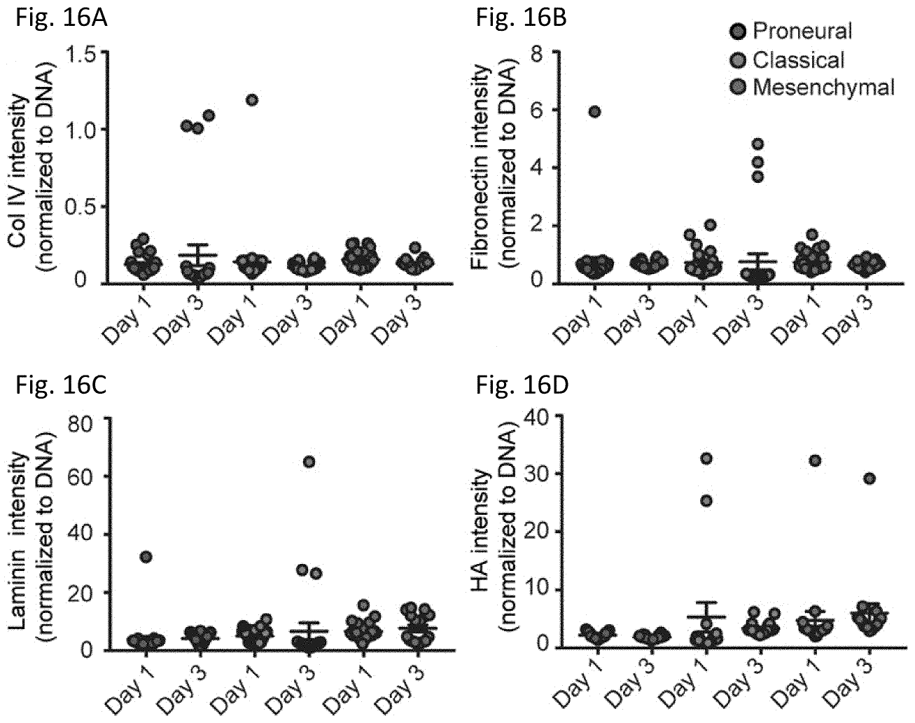

DNA methylation of TAM was analyzed by recovering macrophages and GBM cells from the GBM-on-a-Chip culture and performing whole genome DNA methylation analysis to assess the epigenetic modifications of TAM in molecularly distinct GBMs (CL, PN, and MES. Culturing tumor cells with the presence or absence of macrophages in the niche resulted in different epigenetic profiles and vice versa, the presence of GBM cell also altered the DNA methylation signatures of co-cultured macrophages (FIG. 13A through FIG. 13D). Rap1 signaling pathway, a known regulator of T-cell and antigen-presenting cells (Katagiri K et al., Molecular and cellular biology. 2002 Feb. 15; 22(4):1001-15), was upregulated both in co-cultured GBM and macrophage cells, when compared to the GBM cells and macrophages in mono-cultures (FIG. 14A through FIG. 14E). Interestingly, PD-L1 promoter methylation was slightly hypomethylated in mono- and co-cultured GBM cells (FIG. 15). Combined DNA methylation results suggest that the interaction between TAM and GBM might regulate cytotoxic CD8+ T-cell activation via Rap1 signaling particularly in the CL GBM subtype. Also, PD-L1 expression was not epigenetically silenced in the absence of TAM. In addition, the dynamic interactions between the GBM and ECM were examined over 3 days (FIG. 16A through FIG. 16D) but no significant changes were found in the deposition of HA, laminin, collagen IV and fibronectin in different GBM subtypes.

Optimizing Anti-PD-1 Therapy by Co-Targeting TAM CSF-1 Signaling

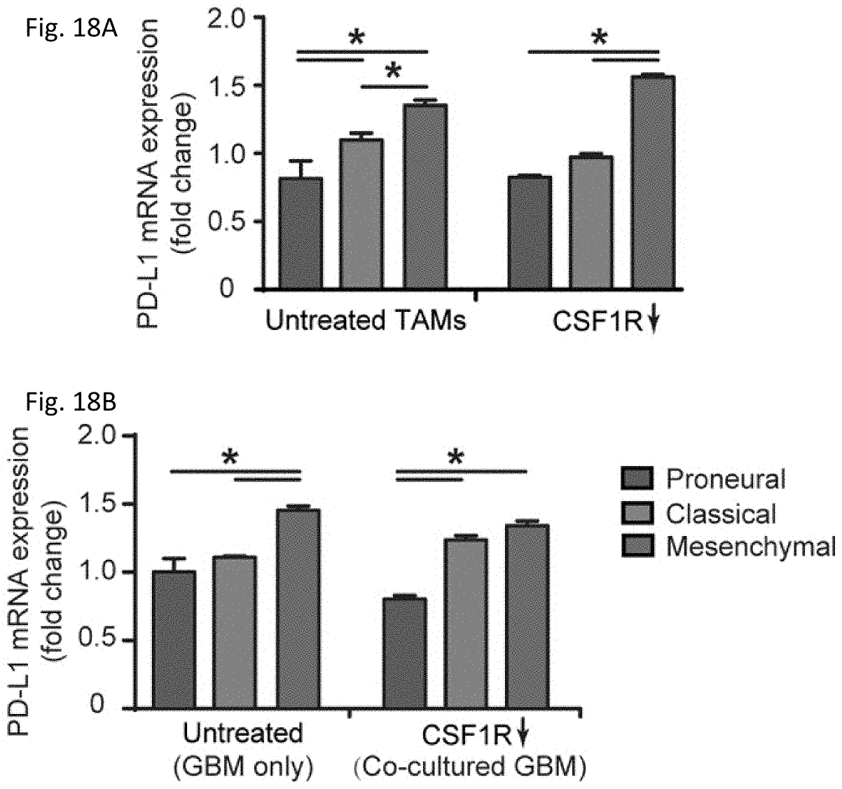

Despite early reports of response to immunotherapy, a recent Phase 3 CheckMate-498 study using PD-1 blockade nivolumab in MGMT—unmethylated newly diagnosed GBM failed to meet primary endpoints, highlighting the need to better stratify patients and identify potential responders as well as testing potential combinational therapies. Using the patient-specific GBM-on-a-chip system, an adjuvant strategy was tested that simultaneously targeted M2-TAM polarization and PD-1 immune checkpoint with nine GBM patient-derived molecularly distinct cell lines (FIG. 17A). Monotherapy or dual-therapy regimens of brain-penetrant CSF-1R inhibitor (BLZ945, 0.1 μg/ml) were delivered to ablate TAM immunosuppressive function and human IgG4 anti-PD-1 monoclonal antibody (nivolumab, 1 μg/ml) to inhibit the PD-1/PD-L1 pathway every 24 hr for 3 days to screen GBM subtype-specific responses. Consistent with previous studies (Pyonteck S M et al., Nature medicine. 2013 October; 19(10):1264; Zhu Y et al., Cancer research. 2014 Sep. 15; 74(18):5057-69), BLZ945 suppressed the polarization of macrophages toward an immunosuppressive M2 phenotype in all three GBM subtypes with more significant CD163 marker suppression in the CL and MES GBM subtypes relative to the PN GBM (FIG. 17B and FIG. 17C). However, BLZ945 treatment alone caused no significant change in PD-L1 expression for both TAM and GBM cells (FIG. 18A and FIG. 18B), implying that BLZ945 monotherapy cannot completely abolish the systemic immunosuppression in the GBM microenvironments.

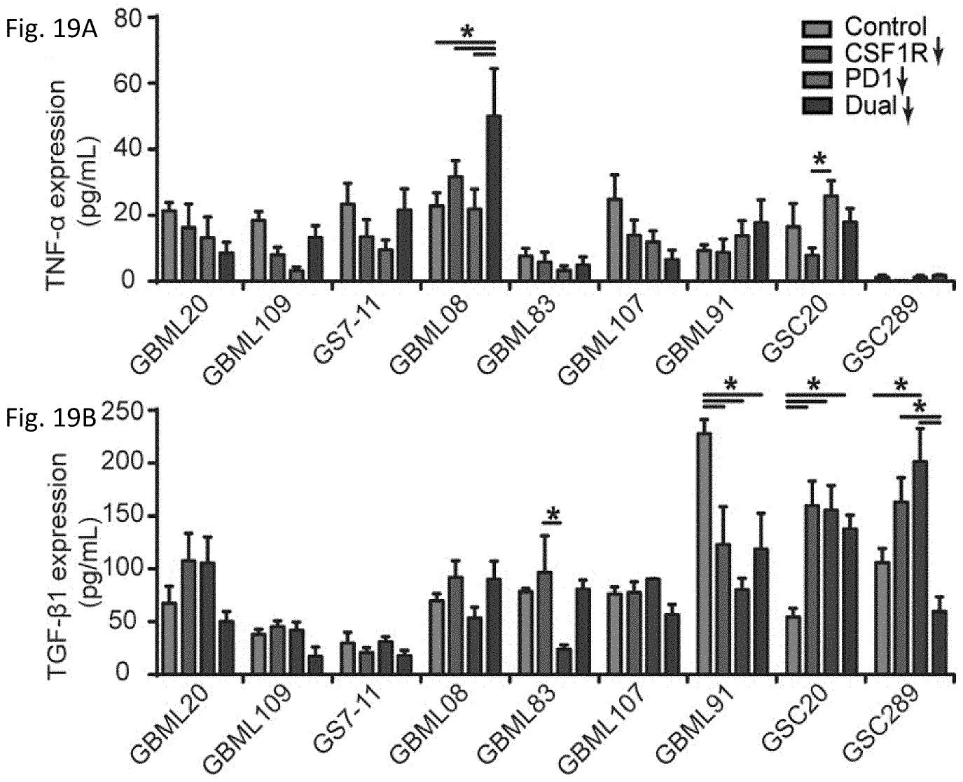

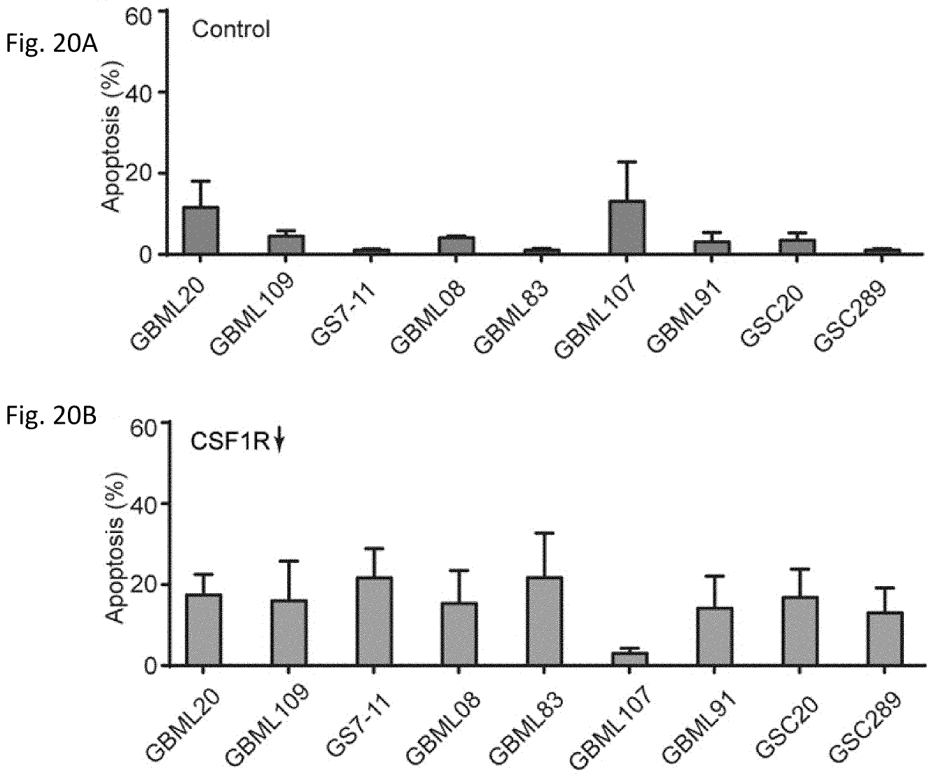

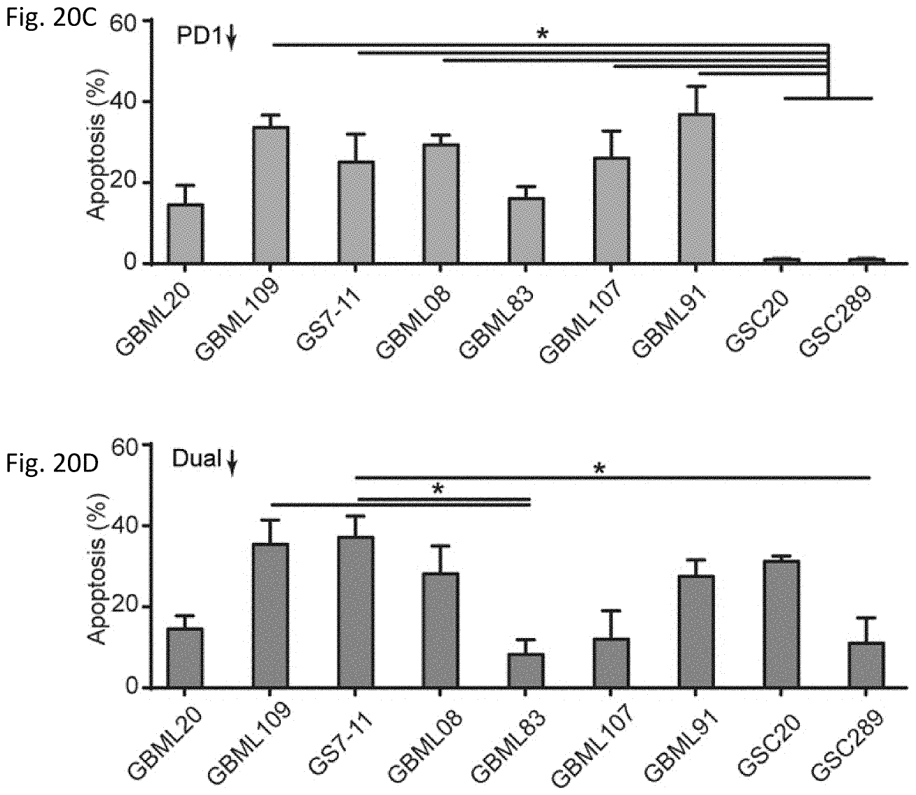

Allogeneic CD8+ T-cell extravasation was examined in the different molecular subtypes of GBM tumors under control (vehicle), BLZ945 or nivolumab monotherapy, and ‘dual’ BLZ945 and nivolumab therapy regimens. The results indicated that CSF-1R blockade can significantly enhance allogeneic CD8+ T-cell extravasation across brain microvessels, compared to PD-1 blockade alone (FIG. 17D). However, as demonstrated by profiling CD154 expression, targeting TAM with BLZ945 alone did not significantly reverse the immunosuppression onto the cytotoxic CD8+ T-cell, compared to the untreated condition (FIG. 17E). In addition, PD-1 inhibition alone did not increase the extravasation of allogeneic CD8+ T-cell in GBM-on-a-Chip but did enhance CD8+T-cell activation in the PN and MES GBMs. PD-1 and CSF-1R dual blockade increased the extravasation of allogeneic CD8+ T-cell across brain microvessels (FIG. 17D and FIG. 17E), reversed the immunosuppression onto CD8+ T-cell in terms of TNF-α and TGF-β1 production (FIG. 17F; FIG. 19A and FIG. 19B), and augmented CD8+ T-cell cytotoxic function with higher GBM tumor apoptosis shown by caspase-3/7 activation (FIG. 17G through FIG. 17H; FIG. 20A through FIG. 20D) for each GBM subtype, specifically the MES GBM, compared to monotherapies.

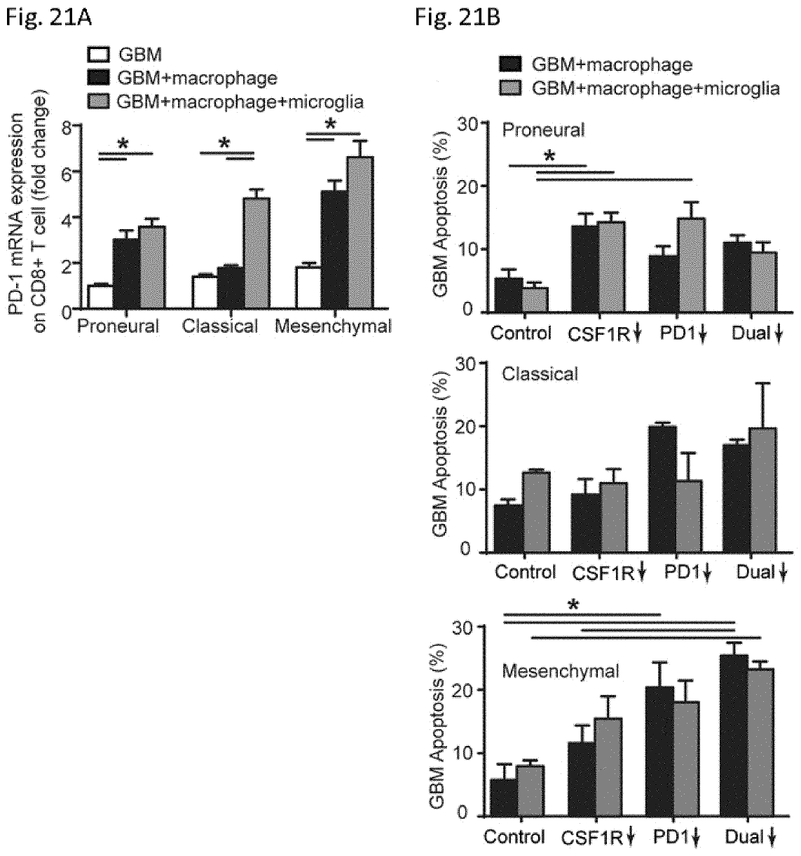

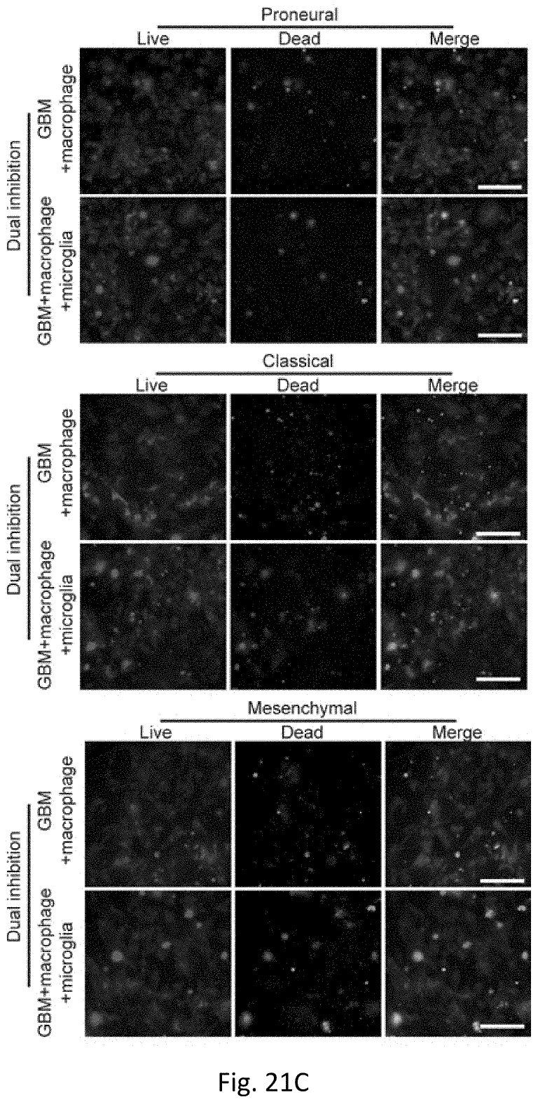

In the brain microenvironment, microglia are the brain-resident macrophages and can play a similar role or cooperate with blood-borne macrophages to regulate brain tumor development and therapy response. The results showed that the presence of microglia in the GBM microenvironment could promote the PD-1 expression on allogeneic CD8+ T-cell (FIG. 21A), but there was no significant change observed in GBM cell apoptosis response to the PD-1 and CSF-1R dual blockade treatment compared to the macrophage only system (FIG. 21B and FIG. 21C). Altogether, pre-clinical screening in the biomimetic GBM-on-a-Chip demonstrated that co-targeting M2-TAM could serve as a potential combinational therapy strategy for improving anti-PD-1 immunotherapy.

Rapid progression, a lack of robust clinical biomarkers, and an insufficient clinical response present major challenges for adapting PD-1 checkpoint-based immunotherapy for GBM patients. GBM patients are largely stratified into clinical trials based on MGMT promoter methylation and IDH1/2 mutation status. However, this does not sufficiently reflect the significant inter- and intra-tumoral heterogeneity. A diversity of genetic and immune signatures of patients in response to PD-1 immunotherapy have been reported, but many of these only integrated genomic and transcriptomic readouts of GBM tumors (Venteicher A S et al., Science. 2017 Mar. 31; 355 (6332)) and nivolumab treatment (Schalper K A et al., Nature medicine. 2019 March; 25(3):470-6; Riaz N et al., Cell. 2017 Nov. 2; 171(4):934-49). The sclinical and experimental data demonstrated that GBM patients of transcriptionally defined subtypes have distinct epigenetic and immune signatures that may lead to different immunosuppressive mechanisms. Nevertheless, these integrated genetic analyses and cell markers from patient biopsies cannot fully capture the dynamic evolution of the tumor microenvironment in response to therapy (Riaz N et al., Cell. 2017 Nov. 2; 171(4):934-49), which may partly account for the limited clinical success of anti-PD-1 therapy.

In the current study, clinical needs are addressed by engineering a human ‘GBM-on-a-Chip’ microphysiological system to dissect the heterogeneous immunosuppressive GBM microenvironments with a real-time and longitudinal analysis of immune activities under different therapy strategies. This work differs substantially from previous methods (Jenkins R W et al., Cancer Discov 8 (2): 196-215; Neal J T et al., Cell. 2018 Dec. 13; 175(7):1972-88), in the aspect of engineering a humanized ex vivo model using patient-derived cells and the capability of real-time monitor of tumor-immune-vascular interactions and therapy responses for screening optimized PD-1 blockade. Importantly, the GBM-on-a-Chip allows for a multidimensional readout of patient-specific responses to different immunotherapy regimens ex vivo on the basis of cellular (immune cell infiltrate composition, phenotypes, and dynamics), epigenetic, transcriptomic, and secretomic signatures to examine the prognostic relationship between patient response and the GBM subtypes (PN, MES, and CL) and genetic mutations (IDH). The results revealed that different subtypes of GBMs illustrated distinct CD8+ T-cell kinetics as allogeneic CD8+ T-cell extravasate across the 3D brain microvessel, traverse through brain-mimicking tissue, and interact with TAM and patient-derived GBM cells. Using the GBM-on-a-Chip model, M2-like CD68+CD163+ TAMS were demonstrated to dominate the immunosuppressive microenvironment in the MES GBM by restricting the dynamics of CD8+ T-cell recruitment and activation, which can be effectively reversed with CSF-1R and PD-1 dual blockade therapy. Moreover, targeting immunosuppressive TAM alone with a CSF-1R inhibitor increased allogeneic CD8+ T-cell infiltration in the tumor, however alone it still yielded a limited effect on tumor apoptosis consistent with previous studies (Quail D F et al., Science. 2016 May 20; 352 (6288)). Similarly, targeting PD-1 alone resulted in a modest effect on allogeneic CD8+ T-cell extravasation. Thus, the findings provide a rationale to combine CSF-1R blockade to optimize the therapeutic effect of immune checkpoint blockade, particularly for the aggressive MES GBM. In the brain microenvironment, brain resident microglia are considered as another source of TAM to regulate brain tumor development and therapy response. The results indicated that microglia in the GBM microenvironment might have a similar immunosuppressive effect with the PBMC-derived macrophages on CD8+ T-cell PD-1 expression and functionality. Yet, no significant change in GBM cell apoptosis response to the PD-1 and CSF-1R dual blockade treatment was observed in the study, which might be contributed by the complex reprogramming of microglia phenotypes in the brain tumor microenvironment as shown previously (Cannarile M A et al., Journal for immunotherapy of cancer. 2017 December; 5(1):1-3).

The GBM model can be modified as an autologous model constructed with all patient-derived cells. An intact blood-brain barrier (BBB) can hinder therapeutically effective drug delivery and limit the drug efficacy in some brain tumors. However, it is well-established that BBB is uniformly disrupted in GBM which leads to leaky blood vessels (Sarkaria J N et al., Neuro-oncology. 2018 Jan. 22; 20(2):184-91). Thus, a simplified GBM microenvironment model without BBB construction can still serve as a suitable and useful model to dissect the GBM tumor-immune-vascular interactions ex vivo.

Altogether, the feasibility of a patient-specific screening for immunotherapy responses ex vivo was demonstrated with the GBM-on-a-Chip platform to dissect the heterogeneous tumor immune microenvironments, rationalize and screen effective therapeutic combinations and facilitate precision immuno-oncology. A truly personalized GBM-on-a-Chip system can significantly accelerate the pace for identifying novel therapeutic biomarkers, developing patient-specific immunotherapeutic strategies, and optimizing therapeutic effect and long-term management for a broader GBM patient population.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A tumor-on-a-chip device, comprising:
a cartridge housing;
a central chamber embedded in the cartridge housing; and
a first plurality of evenly spaced micropillars arranged in a substantially circular shape within the central chamber and a second plurality of evenly spaced micropillars arranged in a substantially circular shape within the first plurality evenly spaced of micropillars, such that the central chamber is partitioned into at least an outer region, a middle region, and an inner region;
wherein each of the outer region, middle region, and inner region is fluidly connected to at least one aperture; and
wherein the outer region comprises endothelial cells configured to mimic a microvasculature, and the middle region comprises tumor cells configured to mimic a tumor.

2. The device of claim 1, wherein the inner region is configured for media perfusion and waste removal.

3. The device of claim 1, wherein each of the cells is an autologous cell.

4. The device of claim 1, wherein the first plurality of evenly spaced micropillars and the second plurality of evenly spaced micropillars are concentric.

5. The device of claim 1, wherein each micropillar of the first and the second plurality of evenly spaced micropillars has a cross-sectional shape selected from the group consisting of: circular, ovoid, square, rectangular, triangular, trapezoidal, and polygonal.

6. The device of claim 1, wherein the first and the second plurality of evenly spaced micropillars are evenly spaced by a distance between about 50 μm and 200 μm.

7. The device of claim 1, further comprising one or more sensors comprising capture molecules or probes positioned within the central chamber.

8. The device of claim 7, wherein each of the capture molecules or probes is selected from the group consisting of: antibodies, antibody fragments, antigens, proteins, nucleic acids, oligonucleotides, peptides, lipids, lectins, inhibitors, activators, ligands, hormones, cytokines, sugars, amino acids, fatty acids, phenols, and alkaloids.

9. The device of claim 7, wherein each of the one or more sensors is positioned between each of the micropillars.

10. The device of claim 1, wherein the device is configured to replicate or mimic a tumor selected from a cancer consisting of: bladder cancer, bone cancer, brain and spinal cord tumors, brain stem glioma, breast cancer, lung cancer, lymphoma, cervical cancer, colon cancer, colorectal cancer, esophageal cancer, gastrointestinal cancer, hepatocellular (liver) cancer, kidney (renal cell) cancer, melanoma, oral cancer, ovarian cancer, and prostate cancer.

11. The device of claim 10, wherein a device configured to replicate or mimic a brain tumor comprises tumor cells that are glioblastoma cells and further comprises tumor-associated macrophages.

12. The device of claim 11, wherein the outer region comprises a population of circulating T-cells.

13. A method of determining anti-cancer treatment responsiveness, comprising the steps of:
providing the device of claim 11;
administering an anti-cancer treatment to the device; and
determining anti-cancer treatment responsiveness based on a measured change in the device.

14. The method of claim 13, wherein the anti-cancer treatment is a chemotherapeutic selected from the group consisting of: temozolomide, procarbazine, cisplatin, methotrexate, carmustine, lomustine, irinotecan, etoposide, carboplatin, vincristine, and cyclophosphamide.

15. The method of claim 13, wherein the anti-cancer treatment is an immunotherapeutic selected from the group consisting of: dinutuximab, pembrolizumab, naxitamab-gqgk, bevacizumab, durvalumab, ramucirumab, cetuximab, nivolumab, and nimotuzumab

16. The method of claim 13, wherein the measured change is a quantity of live and dead tumor cells after 1-3 days treatment or more.

17. The method of claim 13, wherein the measured change is high T-cell motility from the outer region into the middle region, indicating more responsiveness to anti-cancer therapy.

18. The method of claim 13, wherein the measured change is a polarization of tumor-associated macrophage phenotype towards M2-like phenotype, indicating less responsiveness to anti-cancer therapy.

19. The method of claim 13, wherein the measured change is an increase in cytokine levels of TGF-β and/or IL-10, indicating less responsiveness to anti-cancer therapy.


SEQUENCE LISTING (14 sequences)

SEQ ID NO: 1 - protein, 16 aa, synthetic construct
GCRDVPMSMRGGDRCG

SEQ ID NO: 2 - DNA, 18 bp, synthetic construct
  source 1..18: mol_type = other DNA
ctgatgaagggacttgac

SEQ ID NO: 3 - DNA, 17 bp, synthetic construct
  source 1..17: mol_type = other DNA
ctacagcttgaacatgc

SEQ ID NO: 4 - DNA, 20 bp, synthetic construct
  source 1..20: mol_type = other DNA
caggaaaccagtcccaaaca

SEQ ID NO: 5 - DNA, 20 bp, synthetic construct
  source 1..20: mol_type = other DNA
agcgacctcctccatttacc

SEQ ID NO: 6 - DNA, 20 bp, synthetic construct
  source 1..20: mol_type = other DNA
cgtggcctatccactcctca

SEQ ID NO: 7 - DNA, 20 bp, synthetic construct
  source 1..20: mol_type = other DNA
atcccttgtcccagccactc

SEQ ID NO: 8 - DNA, 20 bp, synthetic construct
  source 1..20: mol_type = other DNA
aaatggaacctggcgaaagc

SEQ ID NO: 9 - DNA, 20 bp, synthetic construct
  source 1..20: mol_type = other DNA
gatgagcccctcaggcattt

SEQ ID NO: 10 - DNA, 19 bp, synthetic construct
  source 1..19: mol_type = other DNA
gctgttgttggtctgtctc

SEQ ID NO: 11 - DNA, 20 bp, synthetic construct
  source 1..20: mol_type = other DNA
catgctcttcataatccttg

SEQ ID NO: 12 - DNA, 20 bp, synthetic construct
  source 1..20: mol_type = other DNA
gagtcaacggatttggtcgt

SEQ ID NO: 13 - DNA, 20 bp, synthetic construct
  source 1..20: mol_type = other DNA
ttgattttggagggatctcg

SEQ ID NO: 14 - protein, 8 aa, synthetic construct
GRGDSPCG